(19) United States
(12) Patent Application Publication
(10) Pub. No.: US 2006/0160169 A1
(43) Pub. Date: Jul. 20, 2006

(54) Cell microarray for profiling of cellular phenotypes and gene function

(75) Inventors: Edward Marcotte, Austin, TX (US); Vishwanath Iyer, Austin, TX (US); Andrew Ellington, Austin, TX (US); Jonathon Davies, Dallas, TX (US); Wei Niu, Austin, TX (US); Alex Scouras, Seattle, WA (US); Rommohan Narayanaswamy, Austin, TX (US)

Correspondence Address: CHALKER FLORES, LLP, 2711 LBJ FRWY, Suite 1036, DALLAS, TX, 75234, US

(73) Assignee: Board of regents, The University of Texas System, Austin, TX (US)

(21) Appl. No.: 11/294,624

(22) Filed: Dec. 3, 2005

Related U.S. Application Data
(60) Provisional application No. 60/633,277, filed on Dec. 3, 2004.

(51) Int. Cl. G06K 9/00; C12Q 1/04; C12M 1/34

(52) U.S. Cl. 435/34; 435/287.1; 382/128

(57) ABSTRACT

The present invention includes compositions, methods and systems for analyzing one or more cell characteristics including the steps of depositing one or more spots on a substrate that include one or more cells from a strain collection, contacting the one or more cells with one or more agents and evaluating optically effect of the agent on the one or more cells.

### STATEMENT OF FEDERALLY FUNDED RESEACH

This invention was made with U.S. Government support under Contracts from the National Science Foundation and National Institutes of Health. The U.S. Government owns certain rights in this invention.

### TECHNICAL FIELD OF THE INVENTION

The present invention relates in general to the field of genetic characterization and cellular pathway regulation and, more particularly, to an apparatus, method and system for the direct analysis of compounds using phenotypic characterization of microarrays of genetically distinct cell strains that include strain libraries, combined with automated microscopy, image collection and analysis.

### BACKGROUND OF THE INVENTION

This application claims priority to U.S. Provisional Patent Application Ser. No. 60/633,277, filed Dec. 3, 2005, the entire contents of which are incorporated herein by reference. Without limiting the scope of the invention, its background is described in connection with the regulation, examination, evaluation or dissection of biological pathways and agents affecting those pathways.

Cellular function is directed generally by the information embodied in the nucleic acid genome (i.e., genotype) through the expression of various genes in the genome of an organism and regulation of the expression of those genes. The cellular characteristics (e.g., phenotype) are defined through the translation of genes into proteins. The determination of gene function and regulation is important for understanding cellular processes, cellular and genetic regulation, cascade reactions and pathways in addition to the identification of potential drug that interact with cellular components.

Typically, identification of a specific gene has been through the expression of the gene associated with a specified phenotype in a model biological system, e.g., a cell or even a transgenic animal. Current methods include the removal of the gene to determine the effect on the phenotype (e.g., morphological, physiological, functional, determinable by an assay or the like). However, gene detection can result in terminal mutations and requires extensive sequence information, requiring enormous monetary and technical resources when prepared on a genome scale. Other methods include the isolation of cDNA of the gene and/or regulatory regions followed by cloning the cDNA into an expression vector and expressing the gene in a cell. This method is also labor intensive and provides little information on gene regulation. The DNA isolation methods are generally insufficient for many applications and typically require extensive sequence information, and enormous monetary and technical resources.

Additionally, some analysis of proteins, protein regulation and protein relationships (proteomics) to genes and gene regulation and gene expression are necessary to understand the cellular processes and pathways. The interaction of proteins is crucial to many aspects of cell function. Examples of protein-protein interactions are evident in hormones and receptors interactions, intracellular and extracellular signaling, enzyme substrate interactions, in intracellular protein location, gene regulation, replication, formation of complex structures and in antigen-antibody interactions. The study of proteins, regulation of protein levels and the relationship to gene regulation and expression may lead to diagnosis of disease, development of drug or genetic treatments, or enzyme replacement therapies. Another technique is a proteomics approach to analyzing differential gene expression analysis. The gene expression analysis approach correlates the genetic differences in different biological samples and the different phenotypes observed in their associated cells, tissues, or organisms (e.g., healthy vs. diseased states, wild-type vs. mutated). However, current proteomic approaches use, e.g., 2-dimensional gel electrophoresis and mass spectrometry to study the protein interaction. These approaches are limited by the ability of currently available materials and techniques.

Understanding the normal or pathological state of a population provides information for the prognosis or diagnosis of disease, development of drug or genetic treatments, or enzyme replacement therapies. Therefore, what is needed is a method, apparatus and system to more fully understand cellular pathways and determine potential therapeutics, antibiotic and biologics that effect those cellular pathways.

### SUMMARY OF THE INVENTION

The present invention may be used to regulate, examine, evaluate, diagnose or dissect a biological pathway using the cellular microarray of the present invention by using the cellular microarray depending on the nature of the host cell and known or unknown test samples, compounds or agents. The cellular microarray of the present invention may also be used as part of a method of identifying genes, cellular pathways and agents that affect cellular processes and pathways.

The present invention includes an apparatus, system and method of analyzing one or more cell characteristics by depositing one or more spots on a substrate, wherein the one or more spots include a suspension of one or more cells from a strain collection. The one or more spots of cells have between about 1, 2, 3, 4, 5-10, 10-20, 20-40, 40-50, 50-60, 70-80 or 80 or more cells and have a diameter of between about 100 and 300 μm in diameter. In one embodiment, the one or more spots are about 200 μm in diameter. The disposing of the one or more spots may be performed manually, by an automated system or by a robotic mechanism.

A next step is to contact or treat the cells with one or more agents, followed by the step of evaluating optically the one or more cells, which may include observing the cells with one or more of the following: the light intensity, the fluorescence lifetime, the polarization, a wavelength shift, a wavelength emission a wavelength adsorption, a chemiluminescence emission, FRET emission, one or more ELISA emissions or combinations thereof. The present invention can also include taking an image of the one or more cells either before treatment, during treatment, or after treatment. Additionally, a control image may be taken, wherein the image includes one or more cells before contacting the one or more cells with the one or more agents, followed by a comparison and analysis of genotypic, phenotypic, transcriptional, translational or port-translational changes following exposure, modulation, changes in concentration, location and/or withdrawal of one or more agents, e.g., test agents, pools or combinations of test agents. In certain embodiments, the agents may be extracts, partially purified extracts, mostly purified or purified test agents.

The cells used in the invention may include cell strain collections of any species of microorganism or cells or cell lines derived from multicellular organisms, which include a haploid deletion strain collection. Examples of cells and/or cell lines that may be used with the present invention include, e.g., prokaryotes (bacteria), plant cells (such as pollen) and cell lines derived from multicellular organisms, including animal cells (primary or long-term cell clones, cell lines or mixed populations of cells), human, mouse, rat, yeast, archaebacteria, etc., as will be know to those of skill in the art. Additionally, the strains may include a strain collection that lacks the coding sequence of one or more genes. In one embodiment, the cone or more cells are from a S. cerevisiae haploid deletion collection, although other cell lines may be used. The cell strains include, e.g., the yeasts in the genera of Candida, Saccharomyces, Schizosaccharomyces, Sporobolomyces, Torulopsis, Trichosporon, Tricophyton, Dermatophytes, Microsproum, Wickerhamia, Ashbya, Blastomyces, Candida, Citeromyces, Crebrothecium, Cryptococcus, Debaryomyces, Endomycopsis, Geotrichum, Hansenula, Kloeckera, Kluveromyces, Lipomyces, Pichia, Rhodosporidium, Rhodotorula, and Yarrowia.

The optical evaluation may include observing the cellular characteristics, e.g., cell morphology, cell physiology, cell shape, cell structure, cell death, cell division, cell auxotrophy, the presence of a component, the presence of a nucleic acid sequence or combinations thereof. Furthermore, the step of evaluating optically may include recording one or more properties observed in the one or more cells. The recording may be a digital or an analog signal or combinations thereof. In some instances, the one or more properties observed may be e.g., a differential interference contrast spectrum, a fluorescence wavelength, a visible wavelength, cell morphology, cell biochemistry or combinations thereof. The one or more properties may be observed with the aid of one or more agents that may include, e.g., antibodies, probes, stains, nucleic acid stains, antibody stains, affinity reagents or combinations thereof.

The present invention also includes the use of one or more agents that affect one or more pathways, e.g., metabolic, epigenetic, genetic, signal transduction, transcription, transfection, replication, mitosis, meiosis, intracellular transport, extracellular transport, cytoskeletal, oxidative phosphorylation, phosphorylation, locomotion, phagocytosis, RNAi or combinations thereof. These agents may be small organic or inorganic molecules, proteins, peptides, oligonucleotides, aptomers, thioaptomers, vectors, viruses, carbohydrates, antibodies, probes, stains, nucleic acid stains, antibody stains, affinity reagents or combinations thereof.

The present invention also includes the incorporation of one or more reference marks onto the substrate to reference the relative location of the one or more spots, wherein the one or more spots may be referenced and compared. The step of evaluating optically one or more cells may include the step of analyzing one or more cellular features in one or more genetic backgrounds. The optical observation may be of one or more of the following cell characteristics: cell morphology, cell physiology, cell shape, cell structure, the presence of a component, the presence of a nucleic acid sequence or combinations thereof.

The present invention provides a method of detecting defects in an intracellular processes including the steps of depositing one or more spots on a substrate, wherein the one or more spots includes a suspension of one or more cells from a strain collection. The one or more spots of cells have between about 10-20, 20-40, 40-50, 50-60 or 70-80 cells and have a diameter of about 100 to 300 μm. In one embodiment, the one or more spots are about 200 μm in diameter. The strains may include a strain collection that lacks the coding sequence of one or more genes. One embodiment of the present invention also includes the step of analyzing and identifying one or more genes contributing to a specific phenotype(s), identifying one or more genes contributing to one or more specific morphologies, identifying one or more genes contributing to a specific cell behaviors, providing information about the spatial structures controlled by the genes or combinations thereof. The next step includes contacting the one or more cells with one or more substances to be screened for biological effect. The step of contacting may include any method that places the cells in contact with the agent. The one or more substance may include antibodies, probes, stains, a DAPI stain, antibody stains, affinity reagents or combinations thereof. Alternatively, the agents may be small organic or inorganic molecules, proteins, peptides, oligonucleotides, aptamers, thioaptamers, vectors, viruses, carbohydrates, antibodies, probes, stains, nucleic acid stains, antibody stains, affinity reagents or combinations thereof. Followed by the step of measuring the effect of the one or more substances of the intracellular pathway. The effect observed may be e.g., a differential interference contrast spectrum, a fluorescence wavelength, a visible wavelength, a characteristic of cellular morphology or combinations thereof. The method may further include the step of quantifying information relating to the intracellular pathway. One embodiment uses, a haploid deletion strain collection that lacks the coding sequence of one or more genes.

Another embodiment of the present invention includes a method of analyzing gene function. The method includes depositing one or more spots on a substrate, wherein the one or more spots include a suspension of one or more cells from a strain collection. The one or more spots may have between about 5-10, 10-20, 20-40, 40-50, 50-60, 70-80 or 80-100 cells and have a diameter of between about 100 to 300 μm in diameter. In one embodiment, the one or more spots are about 200 μm in diameter. The method includes contacting the one or more cells with one or more agents, followed by imaging the one or more cells and analyzing the one or more cells. Imaging can be performed manually, by an automated system or by a robotic mechanism. The method may also include contacting the one or more cells with one or more agents. Cells for use in the invention include cell strain collections, which include a haploid deletion strain collection. Additionally, the strains may include a strain collection that lacks the coding sequence of one or more genes. The present invention includes the step of analyzing and identifying one or more genes contributing to specific phenotypes, identifying one or more genes contributing to one or more specific morphologies, identifying one or more genes contributing to specific cell behaviors and providing information about the spatial structures controlled by the genes or combinations thereof.

The step of analyzing may include: observing the light intensity, the fluorescence lifetime, the polarization, a wavelength shift, chemiluminescence emission, radiation emissions, FRET emissions, ELISA emissions or other properties that are modulated as a result of the underlying cellular affect the cellular morphology, the cellular physiology, the cell shape, the cell structure, the presence of a component, or combinations thereof as will be known to the skilled artisan.

Another embodiment of the present invention includes a method of analyzing the localization of one or more proteins, including the steps of: depositing a suspension of one or more cells on a substrate, wherein the one or more cells are from a strain collection, imaging the one or more cells and analyzing one or more characteristic of the one or more proteins of the one or more cells. The one or more proteins may include one or more labels or tags. Alternative characteristics for evaluating the cellular microarrays include, e.g., measuring green fluorescent protein (GFP) reporter construct emissions, various enzymatic activity assays, and other methods well known in the art. The cells may also be treated with one or more substances that affect the expressed one or more labeled proteins or proteins that associate with other components. Additionally, the expression may be monitored through the use of the incorporation or association of a label into cellular macromolecules. Macromolecules include, but are not limited to, nucleic acid molecules, sugars, antibodies, vitamins, minerals organic molecules and proteins identified via methods such as those described above.

Additionally, the present invention also includes a method of analyzing the localization of one or more nucleic acid sequences including depositing a suspension of one or more cells on a substrate, wherein the one or more cells are from a strain collection. Next, the image of the one or more cells is analyzed for the one or more characteristic of the one or more nucleic acid sequences within the one or more cells. The nucleic acid sequences may be double stranded or single stranded and may be e.g., DNA, RNA, PNA or combinations thereof. In some embodiments, the nucleic acid sequences may hybridize to one or more labeled sequences. Other embodiments may be monitored through the use of the incorporation or association of a label(s) into cellular macromolecules.

Another embodiment of the present invention is a method of making a cellular microarray including the steps of depositing one or more spots on a substrate, wherein the one or more spots include a suspension of one or more cells from a haploid deletion strain collection. The cell may be from. haploid deletion strain collection cell lines, wherein each strain lacks the coding sequence of a single gene. Additionally, the haploid deletion strain collection may be fixed using any of the methods practiced currently by persons of ordinary skill in the art.

The present invention includes a method of analyzing one or more cell characteristics including the following steps. The first step includes the depositing one or more spots on a substrate, wherein the one or more spots include a suspension of one or more cells from a strain collection. The one or more spots of cells may have between about 10-20, 20-40, 40-50, 50-60 or 70-80 cells and may have a diameter of about 100 to 300 μm in diameter. In one embodiment, the one or more spots are about 200 μm in diameter. The one or more spots of cells may be deposited manually, by an assembly line, by an automated system, or by a robotic system. The next step is to contact the one or more cells with one or more agents. The one or more cells used in the invention include may a cell strain collection, which include a haploid deletion strain collection. Additionally, the strains may include a strain collection that lacks the coding sequence of one or more genes. The present invention can also include taking an image of the one or more cells either before treatment, during treatment, or after treatment.

Additionally, a control image may be taken, wherein the control image is of the one or more cells before contacting the one or more cells with the one or more agents. The next step includes evaluating optically the one or more cells that includes observing one or more characteristics, e.g., the light intensity, the fluorescence lifetime, the polarization, a wavelength shift, a change in absorbance, a change in emissions, combinations thereof and the like.

The optical evaluation includes observation of cell characteristics e.g., cell morphology, cell physiology, the cell shape, the cell structure, the presence of a component, or combinations thereof. Furthermore, the present invention includes the step of evaluating optically which may include recording one or more properties of the one or more cells. The recording may be a digital signal, an analog signal or combinations thereof. In some instances the one or more properties observed include e.g., differential interference contrast spectrum, fluorescence wavelength, visible wavelength, a cell morphology, cell biochemistry or combinations thereof. The present invention also includes one or more agents that affect one or more pathways selected from metabolic, epigenetic, genetic, signal transduction, transcription, transfection, replication, mitosis, meiosis, intracellular transport, extracellular transport, cytoskeletal, oxidative phosphorylation, phosphorylation, locomotion, phagocytosis, RNAi or combinations thereof. These agents may be antibodies, probes, stains, nucleic acid stains, antibody stains, affinity reagents or combinations thereof.

The present invention provides for an automated cell analyzing system including a substrate, wherein the substrate has one or more spots deposited thereon, wherein the one or more spots include a suspension of one or more cells from a strain collection. The system may also include an optical system and a recording system. The optical system may have a microscopy, a stage and an objective. The optical system may also include an automated microscope, a motorized stage, a piezoelectric controlled objective, a camera or combinations thereof. The recording system may be a charge transfer device or a vacuum tube device, a recordable memory device, a digital image, a photographic image, a printed image or combinations thereof. The optical system may include an automated microscope, a motorized stage, piezoelectric controlled objective and a camera. The recording system may be a charge transfer device or a vacuum tube device. The automated cell analyzing system may also include one or more reference marks on the substrate, to reference the relative locations of the one or more spots.

The present invention also provides a method of analyzing gene function that includes the steps of depositing one or more spots on a substrate, wherein the one or more spots include a suspension of one or more cells from a strain collection and contacting the one or more cells with one or more agents, followed by the steps of imaging the one or more cells and analyzing the one or more cells. Additionally, the step of analyzing the one or more cells may include comparing the one or more cells to a standard. The standard may be an image of one or more cells before contacting the one ore more cells with one or more agents, the standard may be a stored image, a printed image or other controls known to persons of ordinary skill in the art. The strain collection may include cells from a single deletion strain, e.g., a cellular microarray of individual members where the members are stable haploid populations of yeast cells.

The present invention also provides for a method of identifying a pattern of one or more cellular responses including the steps of depositing one or more spots on a substrate, wherein the one or more spots include a suspension of one or more cells from a strain collection and contacting the one or more cells with an agent, the agent selectively affecting one or more cellular responses. The method may also include the steps of identifying the one or more cellular responses exhibited by the one or more cells before and after contact with the agent and comparing the one or more cellular responses to identify a pattern of cellular responses exhibited by the one or more cells after the contact, wherein the pattern of the one or more cellular responses is attributable to the agent is identified.

Yet another embodiment of the present invention includes a method of identifying a pattern of one or more cellular responses attributable to a substance including depositing one or more spots on a substrate, wherein the one or more spots include a suspension of one or more cells from a strain collection. Next, the one or more spots are imaged and one or more cellular responses identified. The method may also include the step of contacting the one or more cells with a substance, imaging the one or more spots and identifying one or more cellular responses exhibited by the one or more cells contacted with the substance. Additionally, the method includes comparing the one or more cellular responses exhibited by the one or more cells before the contact and the one or more cellular responses exhibited by the one or more cells after the contact, wherein a pattern of one or more cellular responses attributable to the substance is identified. Another embodiment of the present invention includes the pattern of one or more cellular responses generated by this method.

The present invention also provides an image library of cell morphology including one or more images, wherein the one or more images are of one or more spots deposited on a substrate wherein the one or more spots include a suspension of one or more cells from a strain collection. The image library includes one or more cells contacted with one or more agent that affects cells and one or more uncontacted and/or untreated cells. The one or more images of the image library may be obtained using a charge transfer device (digital), a vacuum tube imaging device (analog) or a combination thereof.

Another embodiment of the present invention is a method of analyzing cell morphology including the steps of depositing one or more spots on a substrate, wherein the one or more spots include a suspension of one or more cells from a strain collection, contacting the one or more cells with one or more agents and imaging the one or more cells and analyzing the one or more cells. The present invention also includes a method of content-based image retrieval that includes the steps of extracting one or more descriptive features from each of one or more images, which represent cell characteristics; the one or more images may be obtained from one or more different measurement tools and recording the one or more descriptive features to form an image collection.

The image collection may also be indexed to produce a searchable database. The imaging method including one or more set of groups based on similar image content and extracting the query image to be characterized, the query image may include one or more descriptive features. Additionally, the method includes the step of retrieving one or more candidate image from the searchable database based on an image similarity criterion to the one or more descriptive features of the query image, and displaying the one or more images. The descriptive features includes: light intensity, fluorescence lifetime, polarization, wavelength shift, chemiluminescence emission, radiation emissions, FRET emissions, ELISA emissions and/or other properties that are modulated as a result of the underlying cellular changes in cell morphology, cell physiology, cell shape, cell structure, the presence of a component, or combinations thereof. The step of analyzing may also includes comparing a property of the one or more cells to a property of a control. The present invention also includes a database of cell characteristics.

Another embodiment of the present invention includes a method of creating a yeast collection. The methods includes depositing one or more spots on a substrate, wherein each of the one or more spots include a unique suspension of one or more haploid deletion cells from a strain collection, wherein the location of each one or more haploid deletion cells are known. A cellular microarray is formed that is used to examine cell characteristics including one or more cells deposited on a substrate, wherein the one or more cells are from a strain collection and are exposed to one or more agents that are placed into contact with the one or more cells and one or more cellular characteristics are produced as a result of the one or more agents placed in contact with the one or more cells evaluated. The substrate may be, e.g., all or at least partially transparent material (e.g., glass, plastic, polymer, or combinations thereof). In some embodiments, the substrate may be coated or doped using e.g., poly L-lysine, ConA, Mn²⁺, Ca²⁺, biotin, streptavidin, carbohydrates, lectins, antibodies and the like or combinations thereof. The strain collection includes a haploid deletion strain collection and each strain in the collection lacks the coding sequence of one or more genes. The cellular microarray includes between about 1-5, 5-10, 10-20, 20-40, 40-50, 50-60,70-80, 80 or more cells in each of the one or more spots. Each of the one or more spots may be between about 100 and 300 μm in diameter. The cellular microarray includes optically evaluating the one or more cellular characteristics. The one or more cellular characteristics being evaluated includes observing: light intensity, fluorescence lifetime, polarization, wavelength shift, wavelength emission, wavelength adsorption, chemiluminescence emission, FRET emission, one or more ELISA emissions or combinations thereof. Additionally, the cellular characteristics observed may be, e.g., cell morphology, cell physiology, cell shape, cell structure, the presence of a component, the presence of a nucleic acid sequence or combinations thereof.

The cellular microarray includes one or more cellular characteristics that are recorded as one or more properties of the one or more cells. The recording may include one or more images recorded as a digital signal, an analog signal or combinations thereof. The one or more properties may include a differential interference contrast spectrum, a fluorescence wavelength, a visible wavelength, a cellular morphology, cellular biochemistry or combinations thereof.

The cellular microarray includes one or more agents that affect the one or more pathways selected from metabolic, epigenetic, genetic, signal transduction, transcription, transfection, replication, mitosis, meiosis, intracellular transport, extracellular transport, cytoskeletal, oxidative phosphorylation, phosphorylation, locomotion, phagocytosis, RNAi or combinations thereof. The one or more agents include antibodies, probes, stains, nucleic acid stains, antibody stains, affinity reagents or combinations thereof.

Another embodiment of the present invention provides a cellular microarray for detecting defects in intracellular pathway including one or more spots deposited on a substrate, wherein the one or more spots comprise a suspension of one or more cells from a strain collection. The strain collection includes a haploid deletion strain collection from various genus and species. The invention also includes one or more substances to be screened for biological effect placed in contact with the one or more cells and an effect of the one or more substance on the intracellular pathway measured. The present invention also provides a yeast cellular microarray collection including one or more spots deposited on a substrate, wherein each of the one or more spots include a unique suspension of one or more haploid deletion yeast cells from a strain collection, wherein the location of each one or more haploid deletion cells are known.

Another embodiment of the present invention includes a cellular microarray for analyzing gene function, which includes one or more spots deposited on a substrate, wherein the one or more spots include a suspension of one or more cells from a strain collection. The present invention includes one or more agents placed into contact with one or more spots and a response of the one or more cells to the one or more agents. The response may include observing the light intensity, the fluorescence lifetime, the polarization, a wavelength shift, other property that is modulated as a result of the underlying cellular affect the cellular morphology, the cellular physiology, the cell shape, the cell structure, the presence of a component, or combinations thereof. Additionally, the response may include identifying one or more genes contributing to a specific phenotype, identifying one or more genes contributing to a specific morphologies, identifying one or more genes contributing to a specific cell behaviors, providing information about the spatial structures controlled by the genes or combinations thereof.

Yet another embodiment of the present invention includes a protein localization cellular microarray for analyzing the localization of one or more proteins including a suspension of one or more cell deposited on a substrate, wherein the one or more cells are from a strain collection coding for one or more proteins, an image of the one or more cells and an analysis one or more characteristic of the one or more proteins of the one or more cells. The one or more proteins may be labeled, e.g., a reporter gene, the incorporation of the label into the protein, the association of a label with the protein, or combinations thereof. Additionally, the label may result from the action of the protein on a substance which then interacts with the label. Furthermore, the cell may be treated with one or more substances that affect the expression of the protein in some manner. In one example, the one or more labeled proteins are GFP-tagged proteins. Additionally, the substance may affect other pathways of the cell including metabolic pathways, epigenetic pathways, genetic pathways, signal transduction pathways, transcription pathways, transfection pathways, replication pathways, mitosis pathways, meiosis pathways, intracellular transport pathways, extracellular transport pathways, cytoskeletal pathways, oxidative phosphorylation pathways, phosphorylation pathways, locomotion pathways, phagocytosis pathways, RNAi pathways or combinations thereof.

Additionally, the cellular microarray for analyzing the localization of one or more nucleic acid sequences includes a suspension of one or more cells deposited on a substrate, wherein the one or more cells are from a strain collection. The cellular microarray includes an image of the one or more cells and an analysis of one or more nucleic acid sequences within the one or more cells.

The use of the present invention includes a cellular response pattern identifying cellular microarray for identifying one or more cellular responses including one or more spots deposited on a substrate, wherein the one or more spots include a suspension of one or more cells from a strain collection with one or more agents placed into contact with the one or more cells, the one or more agents selectively affecting one or more cellular responses. The one or more agents may be contacted with the one or more cells to cause one or more cellular responses and a comparison is made between the one or more cellular responses to identify a pattern of cellular responses exhibited by the one or more cells after the contact, wherein the pattern of the one or more cellular responses is attributable to the agent is identified.

The present invention includes a substance identifying cellular microarray for identifying a pattern of one or more cellular responses attributable to a substance including one or more spots deposited on a substrate, wherein the one or more spots include a suspension of one or more cells from a strain collection and one or more cellular responses exhibited by the one or more cells. One or more substances may be placed in contact with the one or more cells and the one or more spots imaged. Identification may be made of the one or more cellular responses exhibited by the one or more cells contacted with the substance. A comparison of the one or more cellular responses exhibited by the one or more cells before the contact and the one or more cellular responses exhibited by the one or more cells after the contact, wherein a pattern of one or more cellular responses attributable to the substance is identified.

Thus, the present invention uses cell-based assays to identify and/or characterize agents that previously would not have been readily identified or characterized.

### BRIEF DESCRIPTION OF THE DRAWINGS

For a more complete understanding of the features and advantages of the present invention, reference is now made to the detailed description of the invention along with the accompanying figures and in which:

FIG. 1 illustrates certain features of a cellular microarray according to an embodiment;

FIG. 2 illustrates characteristic yeast cell phenotypes observed on cell arrays;

FIG. 3 illustrates results of a cell array-based genome-wide screen for genes participating in the mating pheromone response pathway;

FIG. 4 illustrates the known response pathways;

FIG. 5 depicts cell adherence to a ConA-coated cellular microarray slide surface;

FIG. 6 illustrates a custom-built cellular microarray printing robot;

FIG. 7 is a screenshot of a Cellma annotation database page;

FIG. 8 is an image of aberrant cell morphology phenotypes identified using cell arrays;

FIG. 9 is a graph of grader agreement on shmoo phenotypes;

FIG. 10 illustrates the morphology phenotypes before and after alpha-factor treatment;

FIG. 11 illustrates microtubule immunostaining of cells on a cell array;

FIG. 12 is an image of two typical fields of yeast cells on a cell array;

FIGS. 13A to 13D show yeast cells fixed with formaldehyde, spheroplasted, and printed on a cell microarray;

FIG. 14 showing DIC and GFP-fluorescence images of pheromone-treated cells from the indicated GFP-tagged strains; and

FIGS. 15A and 15B are examples of Escherichia coli spotted cell microarrays (cell chips).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “strain library” is used to describe two or more cells that have been isolated and cloned that have at least one genotypic and/or phenotypic characteristic. The strain library may be a virus, prokaryotic or eukaryotic strain library. In mammals and other higher organism, the strain library may be from a certain tissue (e.g., a strains of liver cells) and/or for a condition (e.g., a lymphoma library). An example of a cell or strain library for use with the present invention is taught in U.S. Pat. No. 6,867,035, issued to Ong is for a cell library or strain library that is indexed to nucleic acid microarrays, relevant portions incorporated herein by reference. For example, a cell or strain library may be constructed by selecting a clone of an ES cell containing a mutation in a gene that is expressed in a cell at one or more desired locations. The cells are indexed to the array organized individual clones having a mutation in an exon fragment, the mutation being in a different exon in cells of different clones and selecting a clone in the collection from which a hybridizing polynucleotide detected is the exon fragment. The exon fragment may be an exon trap vector for mutating, e.g., embryonic stem cells from a mammal.

As used herein, the phrase “capture optically” refers to a method for image processing and devices associated therewith that are designed to perform a variety of functions including the capture, storage, display, processing, evaluating, changing and evaluating one or more images that are captured using any image capture device or substrate. One example of image capture is electronic image captured, wherein the file may be electronically transferred, evaluated, screened, modified and even compared to one or more images. In some instances, the images will be labeled with a one or more coordinated that refer to a specific location of the acquired image to a location on, e.g., an array (e.g., a cell chip) and the image can be stored, compared, evaluated, reconciled, etc. Depending on the level of resolution, each image may include one or more regions that may correspond to one or more pixels depending on the needs of the specific application. The region can be a grid of between about 1 and 1,000 pixels and about 1 and 1,000,000 pixels. In certain embodiments the grids can be: 10 pixels by 10 pixels; 100 pixels by 100 pixels; 1,000 pixels by 1,000 pixels, etc. The intensity and/or color of the image captured may be compared to a longitudinal template, a transverse template, a diagonal template, a custom template or a combination thereof, wherein the comparison of the template to the pattern indicates the presence of a defect or change. In another embodiment, the intensity of the one or more regions is compared to a longitudinal template, a transverse template, a diagonal template and a combination thereof, wherein the comparison of the template to the region indicates the presence of morphological, phenotypic, detectable, labeled or other changes and the like. Furthermore, in some instances the image processing device adjusts the acquisition timing of the digital imaging device in relation to the reflectivity of the surface, the speed at which the digital imaging device is moving relative to the surface or a combination thereof.

The digital imaging device may include a photodiode, charge coupled device, time delay integration device, array of charge coupled devices, time delay integration array of photosensitive elements, film, spectroscopy, infrared, ultraviolet, confocal, fluorescence detection or a combination thereof. Furthermore, the digital imaging device includes a sensor that detects, electromagnetic radiation, laser, UV wavelengths, IR wavelengths, near IR wavelengths, visible wavelengths, sound waves, magnetic fields, radar signals thermal variations and combinations thereof. In addition to one or more digital imaging devices the present invention may have one or more high throughput processing robots that process one or more cell chips or plates that carry the strain libraries or pools of strain libraries. The camera or detection derive may be further connected to one or more positioning devices, chip/plate location reference systems, lights, imagers, wireless network adaptors, Ethernet adaptors, modems, computers, cameras, sensors or a combination thereof in communication with the image processing device. Further additions include a light source; apparatus for controlling the intensity and direction of light according to the level of exposure required by the digital imaging device and optionally one or more reflectors that concentrate the light emitted from the light source on an area of the surface of the cell chip or plate.

As used herein, a “substrate” refers to any solid surface to which the cell strains may be attached or placed. A substrate includes plates and wells that are made from, but not limited to, plastic, glass, nylon, polypropylene, Langmuir-Bodgett films, functionalized glass, germanium, silicon, PTFE, polystyrene, gallium arsenide, gold and silver, gold and silver, e.g., as a plate, a plate with divots, a plate with lumps, on or about well (e.g., a 96-well plate) and the like. Any other material known in the art that is capable of having functional groups such as amino, carboxyl, thiol or hydroxyl incorporated on its surface, is contemplated. This includes planar surfaces, rough surfaces, smooth surfaces spherical surfaces and the like.

As used herein, a “sample” is any mixture of macromolecules obtained from a person. This includes, but is not limited to, blood, plasma, urine, semen, saliva, lymph fluid, meningeal fluid, amniotic fluid, glandular fluid, and cerebrospinal fluid. This also includes experimentally separated fractions of all of the preceding. “Sample” also includes solutions or mixtures containing homogenized solid material, such as feces, cells, tissues, and biopsy samples. Samples herein include one or more that are obtained at any point in time, including diagnosis, prognosis, and periodic monitoring.

As used herein, the expressions “cell” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Different designations are will be clear from the contextually clear.

As used herein the terms “protein”, “polypeptide” or “peptide” refer to compounds comprising amino acids joined via peptide bonds and are used interchangeably. The term “gene” is used to refer to a functional protein, polypeptide or peptide-encoding unit. As used herein, “gene” is a functional term that includes genomic sequences, cDNA sequences or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “sequences” as used herein is used to refer to nucleotides or amino acids, whether natural or artificial, e.g., modified nucleic acids or amino acids. When describing “transcribed nucleic acids” those sequence regions located adjacent to the coding region on both the 5′, and 3′, ends such that the deoxyribonucleotide sequence corresponds to the length of the full-length mRNA for the protein as included. The term “gene” encompasses both cDNA and genomic forms of a gene. A gene may produce multiple RNA species that are generated by differential splicing of the primary RNA transcript.

“Transformation,” as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome.

The term “transfection” as used herein refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell which has stably integrated foreign DNA into the genomic DNA. The term also encompasses cells which transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA.

As used herein, the term “selectable marker” refers to the use of a gene that encodes an enzymatic activity and which confers the ability to grow in medium lacking what would otherwise be an essential nutrient (e.g., the HIS3 gene in yeast cells); in addition, a selectable marker may confer resistance to an antibiotic or drug upon the cell in which the selectable marker is expressed. A review of the use of selectable markers in mammalian cell lines is provided in Sambrook, J., et al., MOLECULAR CLONING: A LABORATORY MANUAL, Cold Spring Harbor Laboratory Press, New York (current edition).

As used herein, the term “reporter gene” refers to a gene that is expressed in a cell upon satisfaction of one or more contingencies and which, upon expression, confers a detectable phenotype to the cell to indicate that the contingencies for expression have been satisfied. For example, the gene for Luciferase confers a luminescent phenotype to a cell when the gene is expressed inside the cell. In the present invention, the gene for Luciferase may be used as a reporter gene such that the gene is only expressed upon the splicing out of an intron in response to an effector. Those cells in which the effector activates splicing of the intron will express Luciferase and will glow.

As used herein, the term “vector” is used in reference to nucleic acid molecules that transfer DNA segment(s) from one cell to another. The term “vehicle” is sometimes used interchangeably with “vector.” The term “vector” as used herein also includes expression vectors in reference to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “detectable labels” refers to compounds and/or elements that can be detected due to their specific functional properties and/or chemical characteristics, the use of which allows the agent to which they are attached to be detected, and/or further quantified if desired, such as, e.g., an enzyme, an antibody, a linker, a radioisotope, an electron dense particle, a magnetic particle and/or a chromophore or combinations thereof, e.g., fluorescence resonance energy transfer (FRET). There are many types of detectable labels, including fluorescent labels, which are easily handled, inexpensive and nontoxic.

As used herein, the term “staining reagent” refers to the overall hybridization pattern of the nucleic acid sequences that comprise the reagent. A staining reagent that is specific for a portion of a genome provides a contrast between the target and non-target chromosomal material. A number of different aberrations may be detected with any desired staining pattern on the portions of the genome detected with one or more colors (a multi-color staining pattern) and/or other indicator methods.

As used herein, the term “labeled” refers to a method to visualize or detect the bound probe, whether or not the probe directly carries some modified constituent. The terms “staining” or “painting” are herein defined to mean hybridizing a probe of this invention to a genome or segment thereof, such that the probe reliably binds to the targeted region or sequence of chromosomal material and the bound probe is capable of being detected. The terms “staining” or “painting” are used interchangeably. The patterns on the array resulting from “staining” or “painting” are useful for cytogenetic analysis, more particularly, molecular cytogenetic analysis. The staining patterns facilitate the high-throughput identification of normal and abnormal chromosomes and the characterization of the genetic nature of particular abnormalities. Multiple methods of probe detection may be used with the present invention, e.g., the binding patterns of different components of the probe may be distinguished—for example, by color or differences in wavelength emitted from a labeled probe.

As used herein, the term “transgene” refers to such heterologous nucleic acid, e.g., heterologous nucleic acid in the form of, e.g., an expression construct (e.g., for the production of a “knock-in” transgenic animal) or a heterologous nucleic acid that upon insertion within or adjacent a target gene results in a decrease in target gene expression (e.g., for production of a “knock-out” transgenic animal). A “knock-out” of a gene means an alteration in the sequence of the gene that results in a decrease of function of the target gene, preferably such that target gene expression is undetectable or insignificant. Transgenic knock-out animals include a heterozygous knock-out of a target gene, or a homozygous knock-out of a target gene.

As used herein, the term “knock-outs” include, e.g., conditional knock-outs, wherein alteration of the target gene can be activated by exposure of the animal to a substance that promotes target gene alteration, introduction of an enzyme that promotes recombination at the target gene site (e.g., Cre in the Cre-lox system), or other method for directing the target gene alteration.

As used herein, the term “knock-in” refers to an alteration in a host cell genome that results in altered expression (e.g., increased or decreased expression) of a target gene, e.g., by introduction of an additional copy of the target gene, or by operatively inserting a regulatory sequence that provides for enhanced expression of an endogenous copy of the target gene. “Knock-in” transgenics include heterozygous knock-in of the target gene or a homozygous knock-in of a target gene and include conditional knock-ins.

The term “stem cell” as used herein refers to pluripotent stem cells, e.g., embryonic stem cells, and to such pluripotent cells in the very early stages of embryonic development, including but not limited to cells in the blastocyst stage of development.

A major goal of biology is finding functions for the many uncharacterized genes in each genome and reconstructing the gene networks underlying cellular and organismal biology. The present invention relates to the characterization of gene function using an array-based platform for automated, high-throughput microscopic imaging of whole cells. Whole cellular microarrays are prepared by printing of cells on microscope slides, then imaged directly or stained for subcellular features, allowing systematic phenotypic characterization of thousands of genetically distinct cells in parallel. In some embodiments, the cells were applied by a robotic printing machine. One embodiment of the cellular microarrays of the present invention is from the collection of approximately 4,800 haploid yeast gene deletion mutants and assayed the role of each gene in determining normal cellular morphology.

The constructed arrays are from the same strains treated with mating pheromone and identified 52 genes whose deletion caused defects in the pheromone response pathway. Sixteen of the known pheromone response genes (e.g., 76% of those expected) are recapitulated, including the mitogen-activated protein kinase signal transduction cascade and new genes implicated in the pathway include ISY1, the transcription factor PAF1, genes controlling vesicular protein sorting and membrane structure, and the uncharacterized genes DFG10, TIR3, GON3, YNRO68C and YDL073W.

The terms “whole-cell microarray,” “cell array,” “cell microarray,” “whole-cell chip,” “cellular microarray” and “cell chip” as used interchangeably throughout the specification to define an array one or more spots on a substrate, wherein the one or more spots include a suspension of one or more cells from a strain collection.

The present invention includes whole-cell microarrays that enable highly parallel, high throughput analyses of cell phenotypes that complement efforts for assessing cell growth and morphology, protein expression levels, and imaging of tissues. However, transfected cellular microarrays or RNAi microarrays are cultured over a microarray spotted with defined DNAs.

The transfected cell microarray is spotted with defined DNAs, allowing transfection of the overgrown cells with different clones, whereas whole-cell microarrays are made by contact deposition of suspensions of cells from an arrayed library onto coated glass slides using a microarray robot.

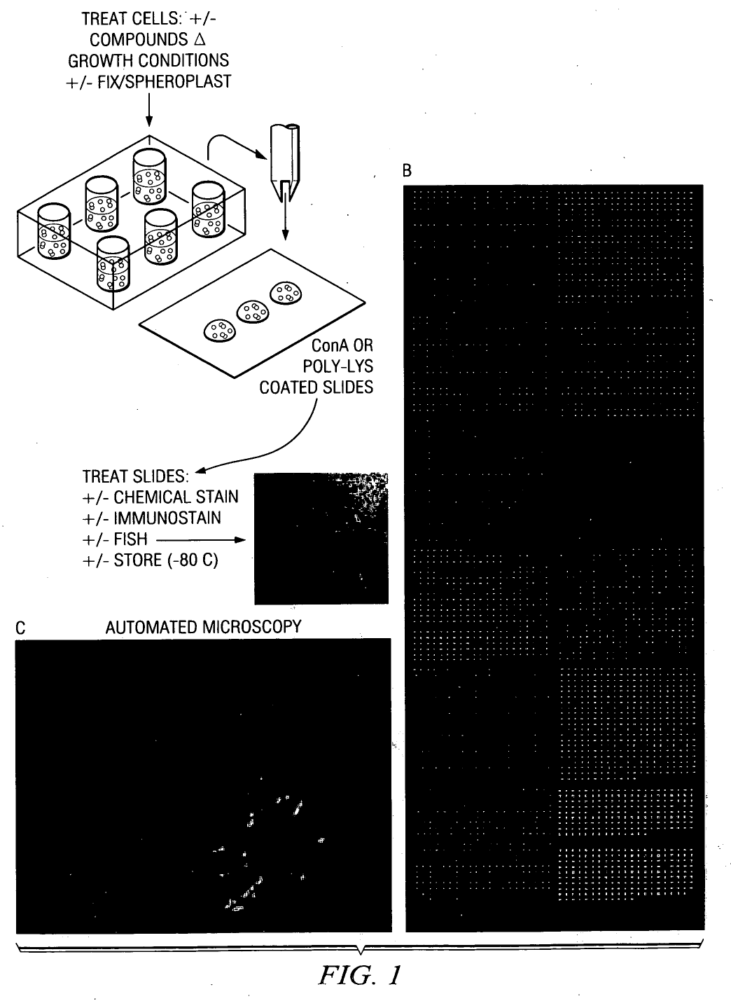

FIGS. 1A to 1C shows an overview of whole-cell arrays. FIG. 1A shows a method of making cellular microarrays using slotted steel pins to robotically print cells from 96 well plates onto poly L-lysine or ConA/Mn²⁺Ca²⁺ coated glass slides. The sample image shows arrayed yeast cells immuno stained for tubulin using FITC-conjugated-goat anti-rat IgG/rat anti-α-tubulin (red), overlaid on a bright field image and a DAPI stained image (blue) of the cells' nuclei. FIG. 1B is a wide-field light scattering image of a cellular microarray (approximately 2 cm×6 cm) containing about 4,800 viable, haploid yeast deletion strains. The bright dots arise from light scattered when scanning the microarray with a Genepix DNA microarray scanner. Spots are about 200 μm in diameter, separated by about 410 μm. FIG. 1C is a close-up of a typical spot from the cellular microarray showing distinct cells at 40×magnification. This image was taken immediately after printing, so growth medium (YPD, 17% glycerol, 200 mg/L G418) is still visible.

The cellular microarrays take advantage of strain collections such as the approximate 4,800 haploid yeast deletion strains, each strain lacking the coding sequence of a single gene. The phenotypes of each of these yeast deletion mutants are characterized in parallel by first printing high-density cell microarrays, each about 200 μm diameter spot containing cells from a distinct deletion strain.

The resulting microarrays were imaged using a microscopy. The high-density format, with all of the strains represented on a single microscope slide, simplifies automated image collection and minimizes reagent use when probing the cells. The present invention allows a single cellular feature to be examined in all approximately 4,800 genetic backgrounds, which allows: the identification of genes contributing to that feature; associating genes with specific phenotypes; and cell behaviors, and providing information about the spatial structures controlled by the genes. Therefore, it allows the effect of agents on cellular pathways to be examined genomically and phenotypically.

Virtually all cell types generate asymmetries of one type or another at their plasma membrane, e.g., prokaryotes, plant cells and cell lines derived from multicellular organisms, including animal cells (primary or long-term cell clones, cell lines or mixed populations of cells), yeast, archaebacteria, etc., as will be know to those of skill in the art. For example, epithelial cells establish junctional complexes between adjacent cells that function as barriers to prevent mixing of apical and basolateral membrane components. In other contexts, such as cell migration and immunological synapse formation, cells are able to generate and maintain highly polarized phenotypes without making use of permanent diffusion barriers. In both cases, membrane microdomains known as lipid rafts are a fundamental component of the polarization process. Lipid rafts are thought to be formed by the tight packing of the long and highly saturated acyl chains of sphingolipids together with sterols. They form platforms for polarized protein delivery and membrane compartmentalization. Different proteins (e.g., glycosylphosphatidylinositol (GPI)-anchored protein) specifically associate with lipid rafts and are thus sorted or retained in a polarized fashion.

Budding yeast Saccharomyces cerevisiae cells exhibit different types of cell polarity. During budding, growth is restricted to a zone adjacent to the previous budding site (haploids) or at either pole of the cell (diploids). First, a hierarchy of positional signals defines the bud site. Then, growth is focused there and restricted to the new bud by a diffusion barrier made of septins that is placed as a collar at the neck between mother and daughter cell. Yeast also exhibit polarized growth during mating. Binding of pheromone, secreted by cells of the opposite mating type, to specific membrane receptors results in the stimulation of a mitogen-activated protein kinase signaling cascade and polarized growth toward the mating partner. Pheromone signaling leads to the arrest of the cell cycle, induction of mating-specific genes and the recruitment of signaling, polarity establishment and cell adhesion proteins to the site of growth. Polarized growth leads to the formation of a mating projection toward the mating partner, thus bringing the cells in direct contact. Then, after the cell wall in the contact zone between both cells is removed, fusion factors promote fusion of the cells. Several fusion mutants have been isolated. Most of the proteins required for cell fusion have been shown to be localized to the mating projection. However, little is known about the mechanisms responsible for the polarization of these proteins. It has been shown that ergosterol-sphingolipid rafts are required for the correct sorting of the major plasma membrane ⁺H-ATPase pump and that lipid rafts are clustered at the tip of the mating projection. Proteins destined to the mating projection partition into lipid rafts and are thus retained and segregated from the rest of the membrane.

Exposure to mating pheromone in haploid cells results in the arrest of the cell cycle, expression of mating-specific genes, and polarized growth toward the mating partner. Proteins involved in signaling, polarization, cell adhesion and fusion are localized to the tip of the mating cell (shmoo) where fusion will eventually occur. The mechanisms ensuring the correct targeting and retention of these proteins are poorly understood. It has been show that in pheromone-treated cells, a reorganization of the plasma membrane involving lipid rafts results in the retention of proteins at the tip of the mating projection, segregated from the rest of the membrane. Sphingolipid and ergosterol biosynthetic mutants fail to polarize proteins to the tip of the shmoo and are deficient in mating. Studies have shown membrane microdomain clustering at the mating projection is involved in the generation and maintenance of polarity during mating.

Therefore, the yeast mating response provides a useful system for the study of cell differentiation, cell secretion and cell fusion. Saccharomyces cerevisiae haploid cells exposed to pheromones stop their progression through the cell cycle, undergo polarized cell growth and form a mating projection, acquiring a pear-shaped morphology called shmoo. Polarized mating cells make contact and attach firmly, forming a prezygote. The cell wall separating the partners must then be degraded and haploid nuclei must merge into a diploid nucleus. A number of genetic screens have identified mutants defective in different steps of mating. Most proteins required for cell fusion localize to the mating projection, but little is known about the mechanisms responsible for their polarization.

The cells were grown on rich media (YPD) and printed onto coated glass microscope slides using a custom-built high-speed robotic arrayer used normally to manufacture DNA microarrays. FIG. 1B shows an image of a cellular microarray printed using this methodology. Furthermore, each spot has 20-40 cells from a single deletion strain, as seen in FIG. 1C using a standard microscope. However, each spot may have about 5-10, 10-20, 20-40, 40-50, 50-60, 70-80 or 80-250 cells from a single deletion strain. The cells on the cellular microarray remain viable and physiologically normal after printing and washing, although cells are typically fixed for imaging purposes. Each cellular microarray is imaged using a fluorescence microscope, in some embodiments the microscope is equipped with a motorized stage and piezoelectric controlled objective, by scanning to each spot, autofocusing and collecting an image using a CCD camera. Differential interference contrast (DIC) imaging was performed to examine the effects of deleting each yeast gene on basic aspects of cellular morphology such as cell shape, size, budding pattern and clumping, from which we expected to find genes controlling fundamental cell growth processes. Systematic analysis of the haploid yeast deletion strain phenotypes on two slides (about 10,000 images) reveals that about 2,000 of the 4,848 strains exhibited atypical morphologies of varying degree.

Two independent graders manually classified phenotypes by severity, penetrance in the population, and type (large, small, elongated, round, and clumped, as well as polarized bud growth and pseudohyphal-like morphology), and 383 strains (8%) were considered to have severe morphology defects. FIGS. 2A and 2B depict characteristic yeast cell phenotypes observed on cell arrays, collected automatically as DIC images at 60×magnification with DAPI stained nuclei superimposed in blue pseudocolor. FIG. 2A shows six phenotypic classes observed among the haploid yeast deletion strains. YIL141W overlaps the AXL2 gene, whose disruption in the deletion strain probably provides the observed morphology. FIGS. 2B shows changes in cell morphology observed after treating the deletion collection with mating pheromone. Many mutants, such as the MRPS5 deletion strain (left), form ‘wild-type’-like mating projections upon adding alpha factor, while cells lacking STE7 (middle) fail to form mating projections, and cells lacking KEL1 (right) form mating projections of unusual morphology.

Genes deleted from strains with a given morphology defect were often functionally diverse. Nonetheless, certain general functions were enriched: elongated strains were enriched (p<0.01) for genes operating in nucleic acid metabolism; cell cycle defects; transcription; and meiosis; large strains were enriched for transporter defects; round strains for cell wall, budding, cell polarity, and cell differentiation genes; small strains for mitochondrial, carbohydrate metabolism, and phosphate transport genes; and strains with polarized bud growth defects for budding, cell polarity, and filament formation genes.

The typical morphology of each haploid deletion strain is verified. The primary morphological differentiation pathway in budding yeast, the response of the cells to the mating pheromone alpha factor during sexual conjugation. Wild-type haploid yeast cells, on detecting pheromone of the opposite mating type via a cell surface receptor, heterotrimeric G protein, and MAP kinase-mediated signal transduction cascade, arrest their cell cycles in G1 phase and grow in a polarized fashion towards the pheromone secreting cells, forming a characteristic cell shape termed a shmoo. Several hundred genes are known to change expression during this process. Contact with shmoos of opposite mating type triggers cell fusion, producing a diploid organism. Although this pathway is well-studied, it has yet to be analyzed to completion.

The entire mating type “a” haploid yeast deletion collection with alpha factor, was then constructed and imaged whole-cell arrays from the fixed and treated cells. Two graders manually examined the cell images for the absence of shmoos. A total of 142 strains appeared to have defects in shmooing, forming either no shmoos or barely detectable shmoos in the imaged fields of cells as seen in FIG. 2B. These 142 strains represent a mixture of genes participating in the pathway and false positive results in the large-scale screen. The 142 strains were retested twice via alpha factor addition and microscopic imaging; 53 of the 142 strains showed consistent shmoo defects. In addition to the high-throughput cellular microarrays screen and the involvement of these genes in the pheromone response pathway, growth assays were conducted measuring the tendency of the strains to arrest growth upon pheromone exposure using the 142 deletion strains plus 271 additional deletion strains as controls with either normal shmooing (wild-type like, as determined from the cellular microarray screen) or enhanced shmooing (marked by increased frequency of shmoos in the cell population).

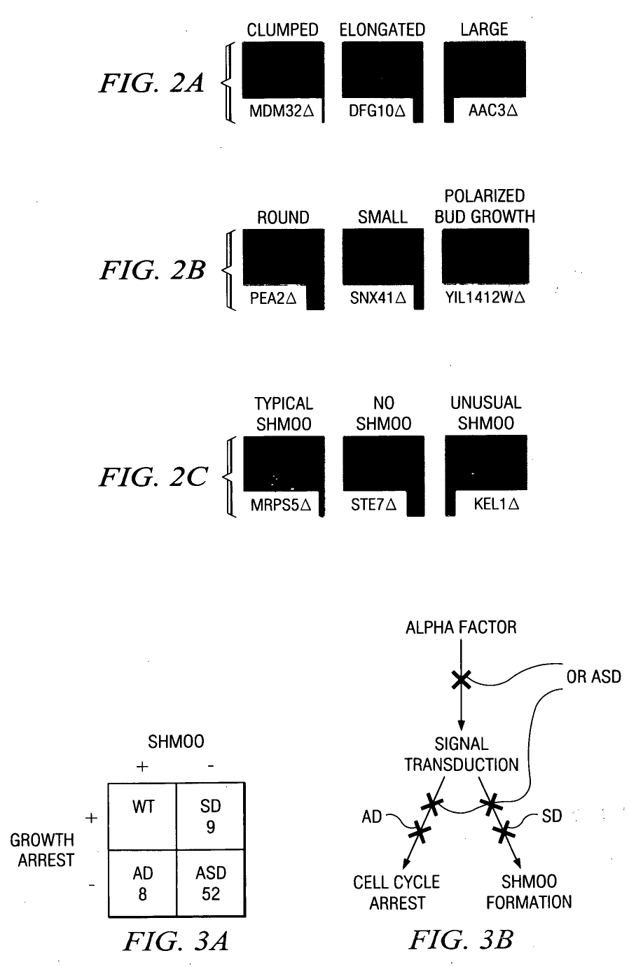

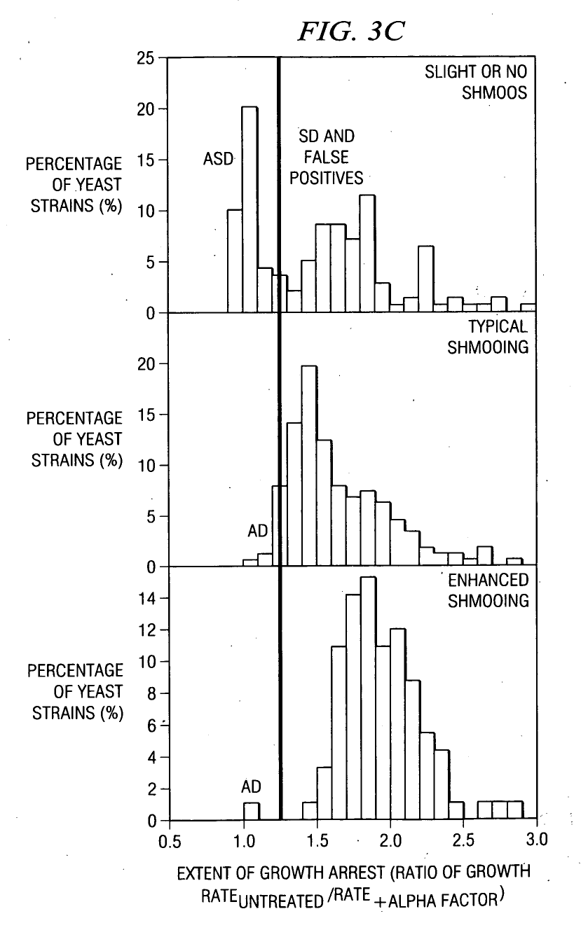

FIGS. 3A to 3C display a summary of the model and graphs of the results of a cell array-based genome-wide screen for genes participating in the mating pheromone response pathway. Strains defective in the mating pheromone response pathway fail to arrest growth when treated with alpha factor, unlike wild-type cells. As two independent phenotypes were screened, three classes of mutants were expected including a true positive alpha factor response pathway mutants (ASD, arrest and shmoo defective), mutants defective only in the shmoo pathway (SD, shmoo defective), and mutants defective only in the growth arrest pathway (AD, arrest defective). FIGS. 3A summarizes the number of genes identified in each category. FIGS. 3B summarizes their interpretation. As only 413 strains were tested in the growth assay, the number of strains with wild-type phenotypes (WT) is omitted. The histograms in FIGS. 3C report the average results of 2 to 3 replicate growth assays for the 142 strains identified from cellular microarrays as failing to shmoo properly, 178 strains forming typical shmoos, and 93 strains forming shmoos with a notably enhanced frequency in the cell population. The true positive alpha factor response pathway mutants (ASD) are clearly separated from non-pathway mutants.

FIG. 3C shows 52 of the 142 strains first identified as shmoo defective also fail to arrest growth upon exposure to alpha factor, as compared to the normal shmooing strains, implicating the deleted genes in the pathway. The lack of growth arrest agreed well with reproducible shmoo defects in 45 of the 53 reproducibly shmoo defective strains also failed to arrest growth. Enhanced shmooing strains arrest even more strongly and appear systematically hypersensitive to the pheromone. The extent of alpha factor-induced growth arrest appears largely uncorrelated with the change in expression of the corresponding genes following alpha factor treatment, even for known genes in the core alpha factor response pathway (data not shown). As two distinct phenotypes were assayed, growth arrest and shmoo formation indicated the possibility of genes defective in either or both pathways a defect in both implicates the gene in the initial alpha factor response pathway or in both downstream pathways, while a defect in only one implicates the gene in the corresponding downstream pathway. Mutants exhibiting both defects, implicated in pheromone detection and signaling were examined and compared to the known pathway.

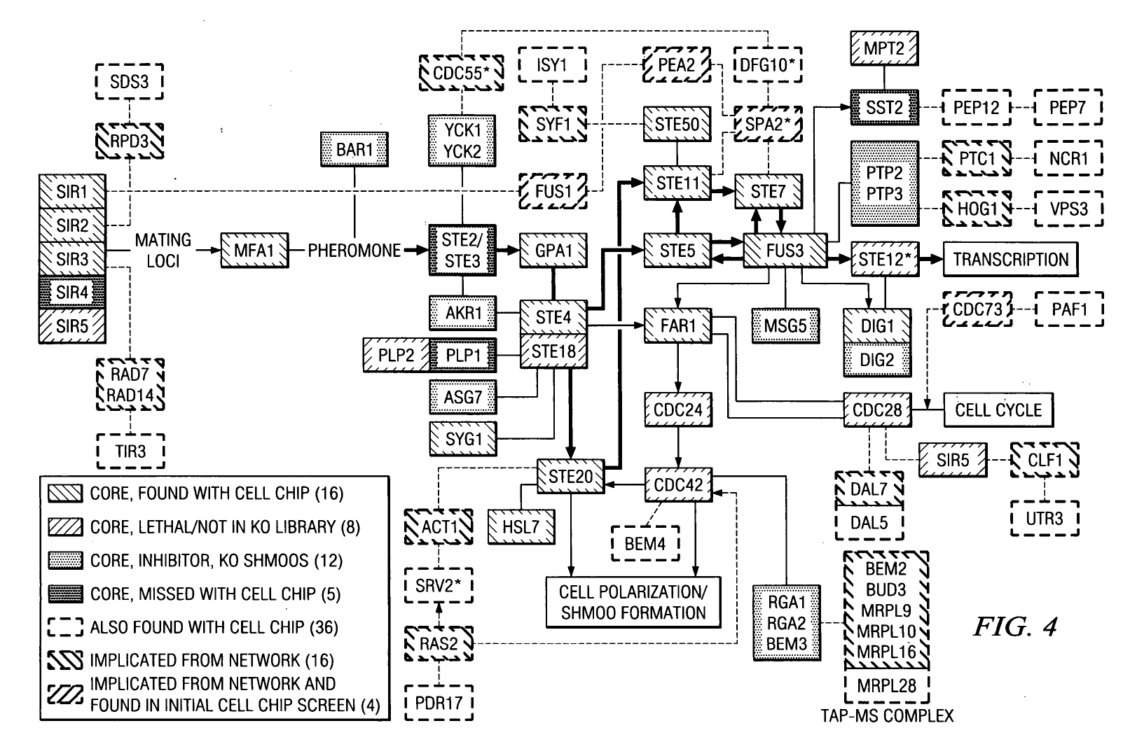

FIG. 4 shows a flow diagram comparison with the known response pathway revealing 21 known genes expected to be found in this screen, 16 were recovered (red labels). Twelve genes are negative pathway inhibitors (blue labels) whose corresponding deletion strains shmoo. Eight essential genes in the pathway (green labels) are absent from the deletion collection. Of the 36 additional genes found, 15 (black labels, boxed) could be associated with the core pathway via protein interactions or mRNA co-expression with intermediates (light gray labels, boxed). Four network-implicated intermediates (light orange labels, boxed) were also found in the initial Cellular microarrays screen, though not reconfirmed as reproducibly shmoo defective. Bold arrows mark the canonical signal transduction cascade culminating in transcriptional changes. Arrows indicate activation; flathead arrows, inhibition; dotted lines, functional genomics linkages. Genes with asterisks are also implicated in filamentous growth.

FIG. 4 shows that of the 41 genes previously known to be in the pathway, sixteen were recovered in the cellular microarray study. Examination of the remaining genes revealed eight genes are not represented in the deletion library (most are essential), twelve genes are negative inhibitors of the pathway and are thus not expected to be observed in this screen, as the deletion strains still shmoo and the remaining five genes were missed for technical reasons related to image focus or low cell count. Thus, of the twenty-one genes expected to be found in this screen, sixteen (76%) were correctly identified, including components of the receptor-coupled heterotrimeric G protein (STE4, GPA1), the MAP kinase signal transduction cascade (STE20, STE11, STE5, STE7, FUS3, FAR1, STE50), silencers of mating loci (SIR1, SIR2, SIR3), and the alpha factor mating pheromone (MFA1).

Additionally, thirty-six genes were found that fail to shmoo and fail to arrest growth upon exposure to alpha factor. Examples include PEP7(VPS19), PEP12(VPS6), and VPS3(PEP6), three genes involved in vesicular trafficking and vacuole protein sorting, suggesting a specific involvement of this system in the pheromone response pathway, possibly related to the role of vesicular trafficking in pheromone receptor localization, endocytosis, or recycling.

There is also a general implication of genes affecting membrane properties, supported by the genes NCR1, involved in sphingolipid metabolism. PDR17, controlling phospholipid synthesis and transport, LAS, controlling glycosylphosphatidylinositol-linked protein transport and remodeling, and ERG4, which catalyzes the final step in ergosterol synthesis and whose deletion produces yeast without ergosterol. Loss of any of these four genes is sufficient to disrupt the pheromone response, possibly indicating sensing of membrane properties feeding back into control of the mating response and consistent with the important role of plasma membrane reorganization in the shmoo response.

These thirty-six genes may be connected to the known pathway (e.g., ‘core set’) using available functional genomics data by searching for the pathways through protein interaction and mRNA co-expression networks that connected the new genes to the core set. Fifteen of the new genes could be reasonably connected to the core set by two interactions or less as seen in FIG. 4. This indicates that these genes may have direct, rather than indirect, roles in the pheromone response pathway. One gene connected in this manner is the transcription factor PAF1, which, with CDC73, forms a distinct complex with RNA polymerase II suggested to regulate transcription of a subset of yeast genes involved in cell wall biosynthesis, but which has pleiotropic effects independent of actively transcribing Pol II. Though PAF1 may control transcriptional events downstream of alpha factor signaling, the growth arrest defect.

In addition to the shmoo formation, PAF1 also works in the main response pathway, or that feedback exists between the downstream shmoo pathway and the cell cycle arrest pathway to couple these responses. Another gene from the screen, ISY1, is similarly pleiotropic but connected to control of the cell cycle, participating in mRNA splicing and the spindle checkpoint. ISY1 exhibits some connection to polarized growth: homozygous diploid deletions of ISY1 exhibit abnormal axial budding. Finally, strains defective in only one of the two assayed phenotypes were identified and implicated in the downstream pathways. The set of genes found that fail to arrest yet shmoo properly was functionally diverse as well as small in size.

Conversely, nine genes were identified exhibiting typical growth arrest yet failing to form shmoos. Interestingly, six of these (VPS8, VPS21, VPS22, VPS23, VPS28, VPS36) are involved in vacuolar protein sorting, with all but VPS8 and VPS21 specific to class E sorting and resulting in inefficient transport out of the endosome, suggesting a critical role of this system in shmoo formation downstream of the pheromone response pathway.

Cellular microarrays have utility beyond morphology screens. Other embodiments of the present invention may be used to analyze the localization of molecules in a cell (e.g., GFP-tagged yeast proteins). Additionally, the present invention may include labels that incorporate into macromolecules including proteins, nucleic acids, compounds or combinations thereof. A variety of cells may be used including cells from any organism and any cell type for which defined libraries of cells can be arrayed. A diverse collection of strains can be arrayed, across a wide variety of genetic backgrounds (e.g., such as other easily manipulated organisms, banks of bacteria and deletion libraries for other microorganisms).

Yet another embodiment of the present invention allows the identification of pathways that are modulated by cellular factors (e.g., alpha factor, transcription factors, messengers, vitamins, minerals, drugs, compounds, toxins, heavy metals, radioactivity and the like). The present invention also allows rapid identification of mutants and pathways that are differentially affected by drugs, compounds, toxins, gene therapy treatments, heavy metals, radioactivity and the like. Additionally, the effect of various drugs, compounds, toxins, gene therapy treatments, heavy metals, radioactivity and the like on the cell may be tested using the present invention.

The cellular microarrays uses a minimal of reagents, samples and cells on the arrays and enables studies such as parallel analyses of cells with different fluorescent antibody probes. Other embodiments include the combinations of the cellular microarrays with automated image processing to produce quantitative strain- and gene-specific data. Another embodiment of the present invention includes a database that includes the functional and statistical data generate and accumulated relating to the genes, ultimately leading to comprehensive, network analyses of cells.

Examples of the cellular microarray construction and imaging. Cellular microarrays were constructed by contact deposition of suspensions of cells from the arrayed collection (e.g., S. cerevisiae haploid deletion strains yeast cells (BY4741 genetic background; MATa his3, leu2, met15 and ura3)) onto a glass slides using a custom-built DNA microarray printing robot. The slide may be ConA or poly-L lysine coated. In about 12 hours, more than a hundred slides can be printed, each containing the entire deletion collection as well as the isogenic wildtype parent strain as a control. Cellular microarrays may be used for imaging immediately after printing or stored at about 4° C. or about −80° C., provided cells are printed with glycerol. Centrifugation may be used to enhance the adherence of the cells to the slide, permitting the slides to be washed before further staining and imaging. The cells may be observed using a microscope and a light source (e.g., visible light, UV light, IR light or fluorescence emissions). Cell images may be collected via an automated microscopy, (e.g., Nikon E800 microscope), a computer-controlled X-Y stage and piezoelectric-positioned objective. The image may be acquired through scanning to the position of each spot and focusing on the spot (e.g., autofocusing). The image is then captured (e.g., a Photometrics Coolsnap CCD camera).

The images and data are stored on a cellular microarray image database (CellMa) for manual examination or automated image analysis. Using a custom modified MetaMorph system (Universal Imaging Corporation) a full set of about 5,000 images may be collected. The slide images from in bright-field mode are ready in about four hours, whereas fluorescent images are ready in about 10 hours.

Example of high throughput screen for alpha factor unresponsive strains. To examine cell morphology phenotypes upon alpha factor stimulation, each yeast deletion strain was sub-cultured into fresh YPD in 96 well Costar tissue culture plates. The cells were then grown for about 36 hours at about 30° C. The cells were then pelleted, and washed with YPD at about pH 3.5, to inactivate the Bar1p protease. Each sample well received 350 μg/ml alpha-factor, which was a concentration sufficiently high to induce shmoo formation in about one half of the cells in the majority of deletion strains. Cells were incubated for about 4 hours at about 30° C. The cells were then fixed with about 3.7% formaldehyde for about one hour at room temperature. The cells were washed with YPD containing about 17% (w/v) glycerol and supplemented with about 20 mM CaCl₂ and about 20 mM MnSO₄. The cells were then spotted onto ConA-coated glass slides. The slides were stained with DAPI and imaged by automated microscopy. The samples were manually scored by two independent graders for extent of shmooing.

Example of an assay of alpha-factor induced growth arrest. The 413 selected deletion strains were grown overnight in YPD. The cells were pelleted, and washed with YPD at about pH 3.5, to inactivate the Barlp protease. The cultures were subsequently split into replicate 96 well plates of YPD both with and without alpha factor at a final concentration of 25 μg/ml wherein maintaining the cells at an optical density at 600 nm (OD600) of about 0.2 to 0.5. Plates were incubated at about 30° C. for about 10 hours and recorded the OD600 hourly from each strain. The cell growth was plotted (e.g., log OD600 versus time) and the slope of each growth curve was calculated from the plot. The effect of alpha factor on the strains was determined as the ratio of the slope from the untreated sample to that of the alpha factor treated sample. The average slope ratios were calculated from 2 to 3 independent assays.

As a result of yeast deletion strains and growth conditions cellular microarrays (e.g., cellular microarrays or cell chips) were manufactured containing all the strains from the S. cerevisiae haploid deletion collection. The set of strains in the BY4741 genetic background (MATa his3Δ leu2Δ met15Δ ura3Δ) generated by the international yeast deletion consortium, in which each strain contains a chromosomal gene replacement of a non-essential gene with a selectable KanMX4 marker that confers resistance to the antibiotic G418. The arrayed library of 4848 such haploid deletion strains was obtained from Invitrogen. Frozen cell cultures in 96-well plates were thawed and used to inoculate 96-well Costar tissue culture plates with 200 μl YPD medium containing the antibiotic G418 (200 mg/L) and 17% glycerol, using a Beckman Biomek FX 96-well pipetting robot to perform all pipetting operations. Copies of the strain collection were incubated for growth at about 30° C., monitoring cell growth by optical density measurement at 600 nm using a 96 well plate reader. Quality control for sterility and cross-contamination was performed by monitoring control wells empty of cells in the master plates. After growth at about 30° C. for about 2 days, copy plates were agitated with a plate shaker, sealed and frozen at about −80° C., with each copy thawed prior to use for printing arrays of cells.

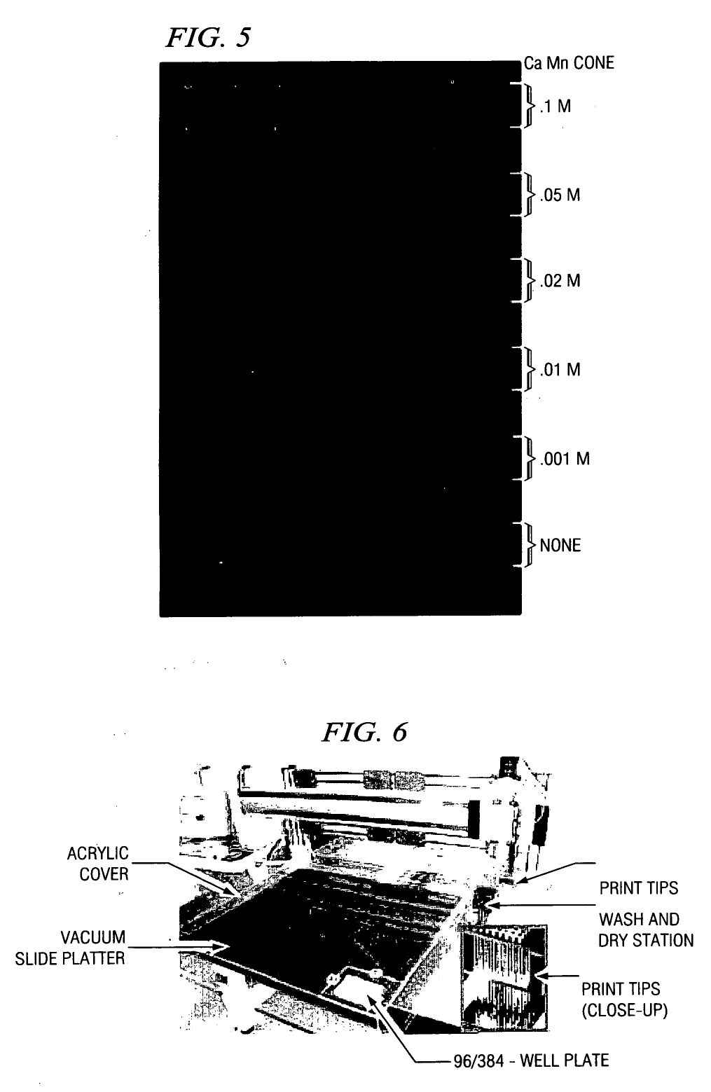

Example of slide preparation poly-L lysine or Concanavalin-A. Coated glass microscope slides were used for all cell arrays. Poly-L-lysine promotes adherence of yeast cells by electrostatic interactions, while the lectin ConA binds to mannose residues in the yeast cell wall. Poly-L lysine coated slides were prepared with an identical protocol as used for DNA microarrays, briefly coating the slide with a 1 mg/ml solution of poly-L-lysine in phosphate buffered saline (PBS) and rinsing in deionized water. Concanavalin-A-coated slides were prepared by incubating slides 15 minutes in a 0.1 mg/ml solution of ConA (Sigma) in PBS, then washing briefly with 95% ethanol. Only freshly prepared slides were used for each print. FIG. 5 displays the use of ConA coated slides required addition of 20 mM CaCl₂ and 20 mM MnSO₄ to each well of cells in order to activate the ConA for binding.

FIG. 6 shows an example of a custom-built cellular microarray printing robot. Printing of cell arrays/Cell microarrays/cellchips were printed by contact deposition of suspensions of yeast cells from the thawed arrayed strain collection onto coated glass slides using a custom-built DNA array printing robot as seen in FIG. 6. Printing was carried out using conically tapered about 1/16 inch diameter stainless steel printing tips with about 0.0015 inch slots (e.g., Majer Precision Engineering; MicroQuill 2000) that are sterilized between print runs. The resulting spots are about 200 μm in diameter, spaced about 410 μm apart. The spots were printed in 12 blocks, with each 21 spots in width. Printing may be carried out with printing tips of different diameters and made of different materials. Additionally, spots may be of different diameters and spacing depending on the particular application.

In a standard print run, the tips are rinsed and vacuum dried 3 times after each loading and printing step, sufficient to prevent carryover of cells from one well to the next, as judged by microscopic inspection of putatively empty spots. Thawed 96-well plates with cell suspensions are agitated gently just prior to placement on the printing robot to ensure a mixed cell suspension. Plates are kept covered at all times before and after printing. During printing, the microtitre plates are kept under a clean acrylic cover at all times except during pick up of the cells. All surfaces, including the vacuum slide platter and the underside of the acrylic dust cover, are sterilized by wiping with 70% ethanol. These procedures ensure that there is no detectable contamination of wells in the plates or of spots on the microarray. After printing, the slides were centrifuged flat at about 1500×g for about 5 minutes in a swinging bucket centrifuge adaptor to promote the adherence of the cells to the slide surface

Cellular microarrays can be imaged immediately after printing or stored a 4° C. or −80° C. for extended periods. To prevent condensation when thawing slides stored at −80° C., frozen slides were rapidly thawed by dipping briefly in room temperature 95% ethanol and, then centrifuged dry in an empty 50 ml conical tube at 600 rpm for 5 minutes.

Scanning of cell microarrays: In one example the cellular microarrays were imaged in two steps: first, the lattice of cell spots was determined using a standard DNA array scanner, then each spot was imaged using an automated microscope. Prior to staining and imaging, each slide is marked with four reference marks using a diamond scribe. Two reference marks are visible at the bottom of the image in FIG. 1C, then the slides are scanned using a microarray scanner (e.g., axon genepix 4000A/B microarray scanner). The spots of cells are detected as bright spots in the 532 nm detector channel because of light scattering by the cells and by the droplet of media or dried liquid at each spot as seen in FIG. 1C. Glycerol present in the medium that cells are suspended in during printing inhibits evaporation and enhances the brightness of each spot. Software (e.g., GenePix scanner software) may be is then used to fit a two-dimensional grid over the spots to define the block, row, and column location of each spot, thus providing an x, y coordinate with each spot in the scanner's system of coordinates. These x, y coordinates are written out to a file (e.g., GenePix GPR-format file), as well as the associated strain identities (stored as a GenePix Gene Array List (GAL) file).

Each slide has an associated set of coordinates describing the relative locations of each cell spot, their identities, and the locations of the reference marks. Spot coordinates can be converted from the GenePix coordinate system to the optical microscope coordinate system through the use of the four reference points and an affine transformation. Slides are then stained or otherwise manipulated prior to microscopy. For typical brightfield or DIC microscopy, slides are washed with water after scanning in order to remove glycerol, dried via about 5 minutes of centrifugation, and a few drops of mounting media are applied containing 100 ng/ml DAPI nuclear stain. Slides are then covered with 24 mm×60 mm cover slips (e.g., 24 mm×60 mm) and sealed (e.g., nail polish). In one embodiment, an automated microscopy was carried out using a Nikon E800 with the CF160 optical system, and outfitted with a motorized X-Y stage with 0.1 micron resolution, a piezoelectric auto-focus device for 9.7 nm focusing resolution, a Photometrix Coolsnap camera with 1392×1040×12 bit pixel resolution, filters for differential interference contrast (DIC), fluorescence and visible wavelengths, and MetaMorph software. First, the reference marks on the slide are located and their positions recorded using the microscope's coordinate system.

Affine transformations provide a useful group of operations that can be applied to reference image features to changes of coordinate system, changes of units of measurement, and referencing scanned images. An affine transformation changes the positions of points in a plane and moves lines into lines, while retaining their intersection properties. The affine transformation can be expressed as a transformation that fixes some special point (e.g., the origin) followed by a simple translation of the entire plane. The transformation can then be represented by matrices of two-by-two arrays of numbers.

An affine transformation matrix is derived that converts coordinates in the GenePix coordinate system to that in the microscope coordinate system, then is applied to all points in the GPR file output from the scanner (e.g., GenePix), creating a MetaMorph format STG file containing the coordinates of all spots converted into the microscope's coordinate system. Images were collected at each spot by executing a MetaMorph “journal” macro at each spot listed in the STG file that autofocused and captured brightfield and fluorescent images, saving each image in TIFF and JPEG format. An entire slide with about 5000 spots can be imaged in about 10 hours, capturing both fluorescent and DIC/brightfield images.

Another embodiment of this invention is an image annotation database. The online relational database developed for warehousing and annotation of cellular microarray images. The present invention includes a database, called Cellma (for Cell MicroArrays), which includes a suite of web pages driven by a relational database (e.g., MySQL relational database). The images may be stored on a central server. In one embodiment the database administrator creates user accounts, enters information about the organisms, strains, and genes studied using the web interface. For data submission, the users first copy images directly to the appropriate location in the directory hierarchy, then create an entry for the slide including name, description, date, preparation, studies, treatments, and other information. Cellma currently supports online manual annotation of high throughput microscopy images.

Although tedious, phenotypes can be reliably scored by visual inspection of images within reasonable timeframes. One study manually graded this way was composed of 5,292 images and took about 20 hours to complete. The images were scored for intensity and penetrance of ten phenotypes and for cell count. Two graders independently scored the images to ensure consistency.

The primary data is stored in the form of image files (one file per spot image) with standardized file names that include the slide name, gene name, treatment, filters used, etc. Files may be saved in both TIFF format, for computational analysis, and in JPEG format, for visual inspection. A person of ordinary skill in the art would realize that other file names protocols and different file types may be used.

In one example all data (e.g., user accounts, slide descriptions and results) is stored in a relational database using the MySQL relational database system (RDBMS) running under Linux. The database has been designed for flexibility in anticipation of other organisms, studies and analysis. Presently there are currently several administration tools, a manual phenotype scoring page, a page to set up and execute automat analyses, and three pages for examining images and results. The administration tools are to manage users, slides, treatments, studies, prints and gene locations.

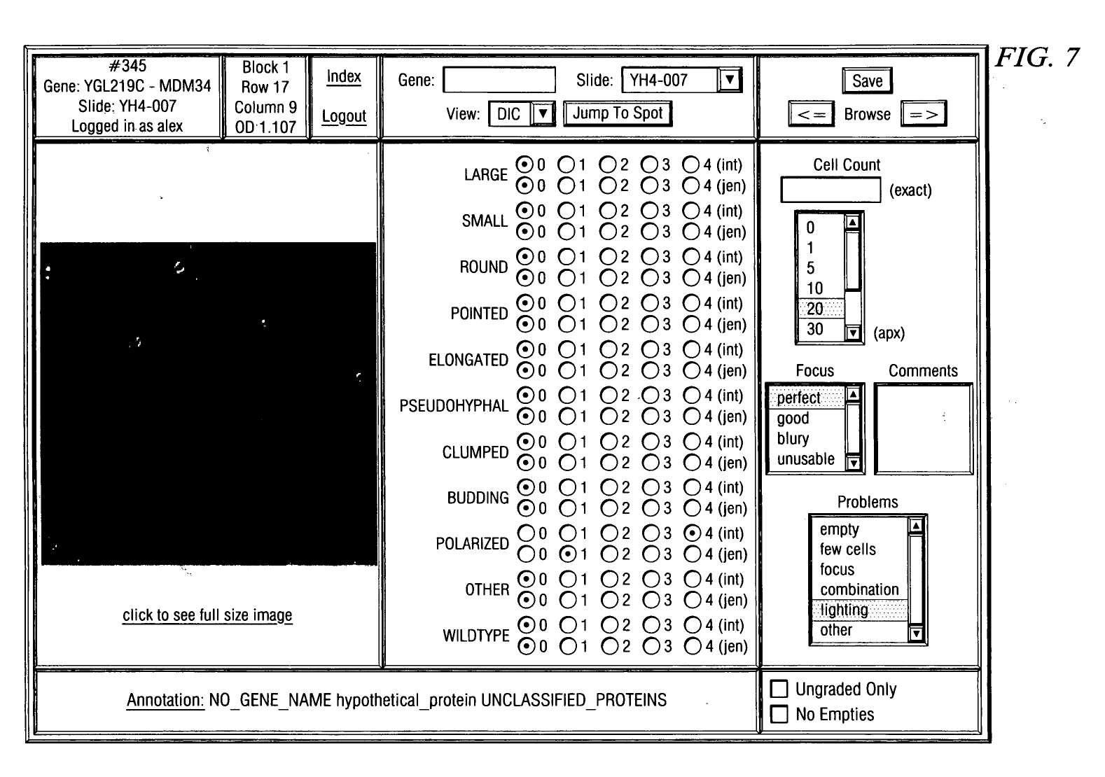

FIG. 7 is a screenshot of a Cellma annotation database page. Morphology phenotypes for each deletion are assigned using a structured annotation scheme. Phenotypes are classified according to their severity and penetrance on a numerical scale, and are specific for a given spot of mutant cells and a given cell array. The present invention provides for scoring phenotypes using a dynamically generated scoring page. Based on studies and treatments applied to a slide, it prompts the grader for intensity and penetrance of each appropriate phenotype as seen in FIG. 7.

The data base model also prompts the user for cell count, focus quality, and problems, and allows the grader to enter comments. The database supports easy navigation between genes, treatments, and slides. Graders can ignore known empty spots and can skip spots and return later. Results of scoring phenotypes can be queried by, e.g., a combination of gene name, study procedure, or mutant phenotype using the web interface.

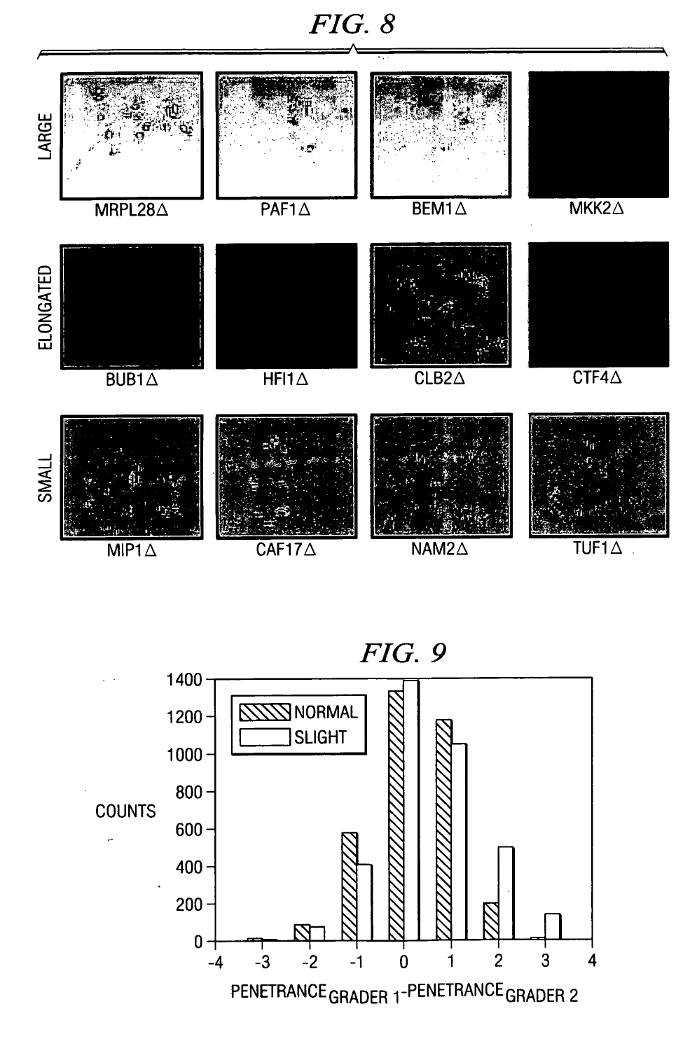

Example of scoring cellular morphology phenotypes. Two graders evaluated independently a set of images from a yeast cellular microarray for strains with atypical morphologies. Phenotypes were scored related to cell size (mutant phenotypes being large or small with respect to the wild-type control), cell shape (mutant phenotypes being round, elongated, pointed with respect to the wild-type ovoid shape) or either a pseudohyphal, clumped or polarized bud growth or other budding defects. FIG. 8 illustrates examples of cell morphology phenotypes. FIG. 8 illustrates aberrant yeast cell morphology phenotypes identified using cell arrays. Each image shows cells deleted for the indicated gene.

Screening for genes affecting the mating pheromone response pathway using cellular microarrays identified genes affecting the intracellular signaling pathway used to respond to the yeast mating pheromone. The pheromone response pathway is a signaling cascade that is activated when yeast cells of opposite mating type are in proximity and secrete mating pheromone. When MATa type yeast cells are treated with the pheromone alpha factor, the activation of this pathway causes G1 arrest and a characteristic “shmoo” morphology. Defects in this signaling pathway prevent shmooing after treatment with alpha factor. Additional protein functions that affect the pheromone response pathway, either directly or indirectly, could be identified by examining cell morphology phenotypes and shmoo phenotypes when the deletion collection was treated with alpha factor.

In one example, the yeast deletion collection was grown to saturation in 96 well plates. Each plate was sub-cultured into fresh YPD in 96 well Costar tissue culture plates and allowed to grow for about 36 hours at about 30° C. without shaking. The plates were spun and washed multiple times in YPD, pH 3.5 to inactivate the Barlp protease. Alpha factor was added to each sample well at a concentration of 350 μg/ml, a concentration sufficiently high to induce shmoo formation in approximately half of the cells in the majority of the deletion strains (and the wild-type control samples) under these conditions. After 4 hours of treatment at 30° C., the cells were fixed in 3.7% formaldehyde for 1 hour at room temperature and washed with YPD containing 17% (w/v) glycerol. At this stage, 20 mM CaCl₂ and 20 mM MnSO₄ were added to each well. The cells were spotted onto pre-cleaned glass slides coated with ConA. While the scoring of phenotypes on these alpha-factor treated cellular microarrays was in progress, the shmoo phenotypes of several hand-picked deletion mutants that had previously been identified as cell morphology mutants in our earlier cellular microarray analyses were examined. The shmoo phenotype of these mutants were compared to that of wild-type cells as well as cells defective for genes known to have a role in the pheromone response signaling pathway.

Examples of manual scoring of shmoo defects. After imaging the alpha factor-treated yeast cells on the cell array, two independent graders scanned visually the set of about 5000 images on the following grading system: The intensity of shmoo phenotypes (e.g., the morphology of the cells) was graded by scoring shmoos into 3 categories: Slight shmoo, Normal Shmoo, or Others, referring to the shapes (degree of shmooing) of the alpha factor treated cells, accompanied by a measure of the abundance of that phenotype across the population of cells imaged, ranging from 1 to 4 (e.g., 0 to 100%). For example, for the concentrations of alpha factor chosen, a normal ‘shmoo’ phenotype (wild type background) had a Normal (2) and Slight (2) indicating that 50% of the cells in the spot had a typical shmoo phenotype, and 50% failed to shmoo. By contrast, a shmoo defective strain would lack any “Normal” or “Other” shmoos, and would be composed of only Slight shmoos; a Normal (4) would indicate an enhanced fraction of normal-looking shmoos in the population, suggesting a hypersensitive response to alpha factor but no change in shmoo morphology. The “other” class of shmoos indicated unusual shmoo phenotypes, such as from bud neck defects. FIG. 9 shows a histogram of the agreement of grades from the two graders-the Gaussian grade distributions indicate that the graders were largely consistent and varied from each other in a stochastic fashion, with no systematic grading bias exhibited, except for an approx. ½ unit higher penetrance on average for grader 1 relative to grader 2.

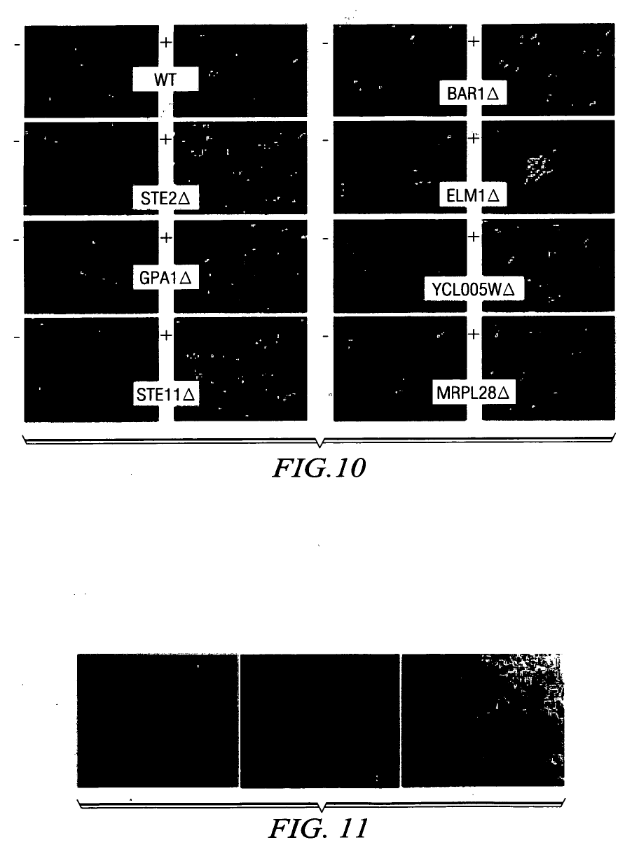

Examples of yeast growth curves +/− alpha factor. To determine if yeast strains arrested growth in the presence of alpha factor, selected strains were picked from the yeast deletion library and grown in YPD overnight until they attained log phase growth. The cultures were spun and washed with YPD pH3.5 to inactivate Bar1p protease. The cultures were subsequently split into replicate 96 well plates, with and without alpha factor at a final concentration of 25 μg/ml, while keeping cells to an OD600 of about 0.2 to 0.5. The plates were incubated at about 30° C. for about 10 hours without shaking and their absorbance was recorded at 600 nm each hour. The slope of each growth curve was calculated from a plot of log OD600 vs. time. The effect of alpha factor on the strains was obtained as the ratio of the slope from the untreated sample to that of the alpha factor treated sample. Average slope ratios were calculated from 2 to 3 independent assays. This analysis, in combinations with the cellular microarray alpha factor treatment analysis, allowed us to identify a number of genes that were affected in their ability to form shmoos after alpha-factor treatment as seen in FIG. 10.

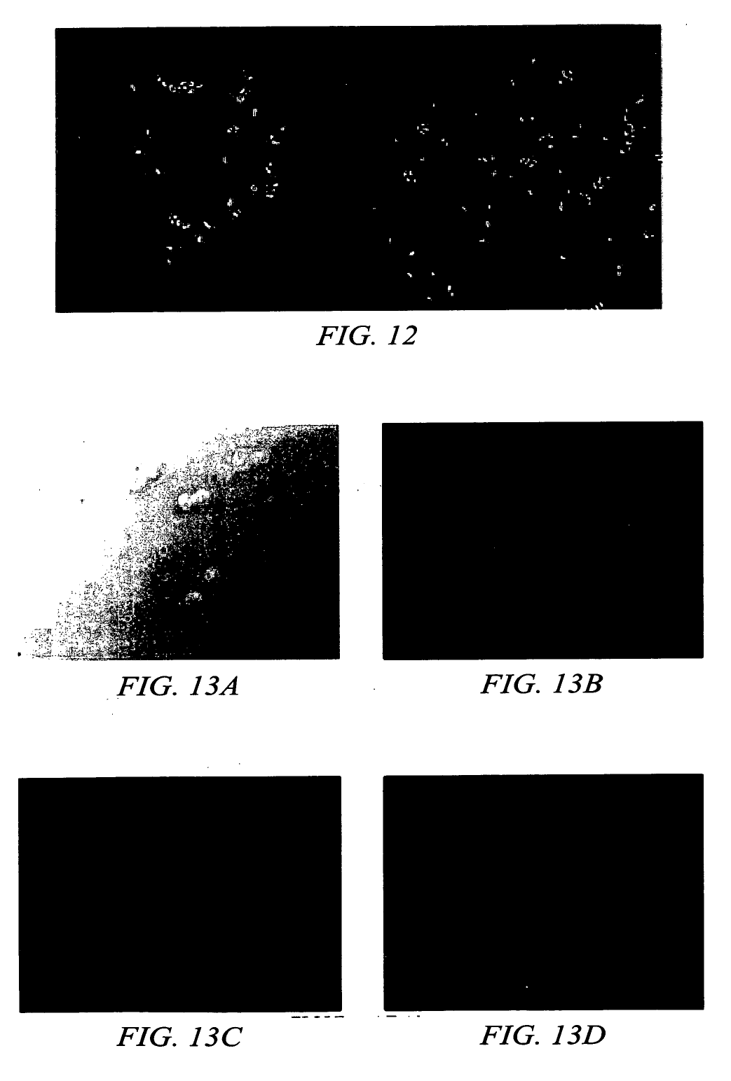

Visualization of molecules on cellular microarrays used cellular microarrays to visualize the localization of macromolecules such as DNA and chromosomes by DAPI staining as seen in FIG. 2 and proteins by antibody staining or other affinity reagents. In one example, microtubules in cells were visualized by probing cellular microarrays with an antibody against tubulin. The cells were first fixed with formaldehyde, spheroplasted and then printed as microarrays on to poly-L-lysine coated slides. Cells on the microarray were permeabilized in cold methanol for five minutes then in cold acetone. Cellular microarrays were probed with rat anti-alpha tubulin (YOL1/34; Serotec) as the primary antibody, followed by an FITC conjugated goat anti-rat 1 gG secondary antibody (Serotec). After washing, the cellular microarrays was additionally probed with DAPI and imaged as before. FIG. 11 shows distinct microtubule morphology using the anti-tubulin antibody, demonstrating that it is possible to perform high-throughput detection of proteins in cells on a cellular microarrays by probing with a specific probe. FIG. 12 is an image of two typical fields of yeast cells on a cell array, collected automatically as bright field images at moderate magnification (40×). Both deletion strains exhibit polarized bud growth defects identified via the cell array images, with the AMD2Δ cells (left) showing high penetrance of the phenotype and the YPR013CΔ cells (right) showing low penetrance.

Table 1 lists examples of the cellular phenotypes observed using cell microarrays.

| Phenotype | Gene deleted |
|---|---|
| Elongate | YMR176W, YMR183C, YCL032W, YKL048C, |
| | YPL024W, YNL215W, YIL093C, YKL101W, |
| | YDR200C, YDR540C, YDL081C, YMR036C, |
| | YBR134W, YNL148C, YIL099W, YBL032W, |
| | YNL095C, YJR099W, YJR145C, YBL091C, |
| | YIL040W, YER120W, YCR094W, YML103C, |
| | YHR010W, YEL007W, YGR262C, YPR119W |
| Large | YPR064W, YGL176C, YKR042W, YJR054W, |
| | YDR528W, YIL090W, YPR198W, YPR045C, |
| | YNL175C, YCL011C, YDR462W, YLR320W, |
| | YLL049W, YBL091C, YDR507C, YLR367W, |
| | YFR013W, YGL164C, YER179W, YJL180C, |
| | YLR192C, YKR059W, YIR003W, YER110C, |
| | YBR054W, YLR390W-A, YFR019W, YGL256W, |
| | YDR252W, YMR070W, YIL161W, YGL173C, |
| | YOR198C, YDL176W |
| Clumpy | YGL259W, YDR349C, YOL001W, YDR388W, |
| | YJL062W, YNL079C, YKL048C, YDR349C, |
| | YBR054W, YER155C |
| Round | YDR034C, YOL001W, YPL138C, YER111C, |
| | YPL191C, YDR388W, YLR319C, YJL062W, |
| | YNL079C, YDL176W, YER109C, YCR087W, |
| | YOR302W, YDR532C, YOR331C, YMR188C, |
| | YOR299W, YOR332W, YBR297W, YER149C |
| Snake/ | YDR453C, YMR246W, YMR058W, YMR191W, |
| Pseudohyphal | YDR242W, YNL148C, YDR200C, YDR540C, |
| | YDL081C, YOL148C, YCR002C, YBR111C, |
| | YOR184W |
| Budding | YNR025C, YDR507C |
| Other | YBR285W, YLR390W-A |

Table 2. Examples of Cells that may be used in an Array.

Enriched Functional Categories in Strains with Defective Morphology

Only 383 strains with “severe” morphology defects are included (i.e., total grader score>=8) All significant functional annotations (p-value<0.01) are shown, as calculated with FunSpec, Robinson et al., BMC Bioinformatics. 2002 Nov. 13;3(l):35)

| | | |
|---|---|---|
| Category | p-value | In Category from Morphology Mutants |

Elongated, 111 genes
MIPS Functional Classification

| | | |
|---|---|---|
| meiosis | 0.00883 | XRS2 DMC1 HOP2 NAM8 RAD50 BFR1 |

MIPS Phenotypes

| | | |
|---|---|---|
| Nucleic acid | 1.92E−05 | PAF1 RAD18 XRS2 CHD1 DMC1 HOP2 KEM1 SRB2 SPT10 |
| metabolism defects | | EST1 SPT21 RAD14 RAD50 CTF4 |
| other cell cycle | 0.002864 | DMC1 HOP2 KEM1 DBF2 HOF1 MIH1 RAD50 WHI2 BFR1 |
| defects | | CTF4 |
| Transcriptional | 0.009708 | PAF1 CHD1 SRB2 SPT10 SPT21 |
| mutants | | |

MIPS Protein Complexes

| | | |
|---|---|---|
| MRE11/RAD50/X | 0.000873 | XRS2 RAD50 |
| RS2 complex | | |
| Non-homologous | 0.00584 | XRS2 RAD50 |
| end-joining | | |
| apparatus | | |

GO Molecular Function

| | | |
|---|---|---|
| single-stranded | 0.000743 | RAD18 DMC1 EST1 |
| DNA binding | | |
| [GO: 0003697] | | |
| Pol II transcription | 0.002839 | PAF1 CHD1 RTF1 |
| elongation factor | | |
| [GO: 0016944] | | |
| transcription | 0.004583 | PAF1 CHD1 RTF1 |
| elongation factor | | |
| [GO: 0003711] | | |
| double-stranded | 0.007699 | DMC1 RAD50 |
| DNA binding | | |
| [GO: 0003690] | | |

GO Biological Process

| | | |
|---|---|---|
| transcription from | 0.000417 | PAF1 CHD1 RPB9 RTF1 SRB2 SPT10 SPT21 FKH2 CAF120 |
| Pol II promoter | | HTZ1 GAC1 EAF3 |
| [GO: 0006366] | | |
| protein-vacuolar | 0.00489 | AUT7 VPS64 VPS75 CVT9 VPS69 VPS66 |
| targeting | | |
| [GO: 0006623] | | |
| nucleobase, | 0.000533 | PRS4 NUP170 PAF1 RAD18 XRS2 CHD1 DMC1 RPB9 KEM1 |
| nucleoside, | | RTF1 PRP18 SRB2 NAM8 IMP2′ SGN1 RTT101 SPT10 PBS2 |
| nucleotide and | | MUD2 MMS22 RSC2 NUP188 SPT21 RAD14 RNH1 FKH2 |
| nucleic acid | | RAD50 CAF120 HTZ1 GAC1 EAF3 CTF4 |
| metabolism | | |
| [GO: 0006139] | | |
| DNA metabolism | 0.000868 | RAD18 XRS2 CHD1 DMC1 IMP2′ RTT101 SPT10 MMS22 RSC2 |
| [GO: 0006259] | | RAD14 RNH1 RAD50 HTZ1 EAF3 CTF4 |
| mRNA splice site | 0.001726 | NAM8 MUD2 |
| selection | | |
| [GO: 0006376] | | |
| biogenic amine | 0.002367 | PRS4 TRP5 EPT1 |
| metabolism | | |
| [GO: 0006576] | | |
| biogenic amine | 0.002367 | PRS4 TRP5 EPT1 |
| biosynthesis | | |
| [GO: 0042401] | | |
| amino acid | 0.002839 | PRS4 TRP5 EPT1 |
| derivative | | |
| biosynthesis | | |
| [GO: 0042398] | | |
| amino acid | 0.002839 | PRS4 TRP5 EPT1 |
| derivative | | |
| metabolism | | |
| [GO: 0006575] | | |
| cell growth and/or | 0.00346 | YBL064C PRS4 AUT7 NUP170 BAP2 AAC3 PAF1 MAL32 |
| maintenance | | RER1 SRO9 CDC10 RAD18 DLD1 VPS64 XRS2 ACN9 TIR1 |
| [GO: 0008151] | | CHD1 DMC1 TRP5 HOP2 ERV14 RPB9 KEM1 RTF1 PRP18 |
| | | DBF2 MAL11 RPL27A SRB2 NAM8 EPT1 KEL1 RPN10 RPI1 |
| | | IMP2′ SGN1 DAL3 RTT101 SPT10 PBS2 PFD1 ATP12 STR2 |
| | | RPL14A MUD2 JEN1 EST1 MMS22 RSC2 NUP188 HOF1 MIH1 |
| | | APG16 SPT21 RAD14 RNH1 ZRC1 FAA4 FKH2 RPL42A VPS75 |
| | | RAD50 CAF120 HTZ1 WHI2 GAC1 BFR1 ODC2 ECM23 EAF3 |
| | | RPL43A CVT9 VPS69 CLB2 CTF4 VPS66 |
| DNA repair | 0.003711 | RAD18 XRS2 IMP2′ MMS22 RAD14 RAD50 CTF4 |
| [GO: 0006281] | | |
| RNA elongation | 0.005281 | PAF1 CHD1 RTF1 |
| from Pol II | | |
| promoter | | |
| [GO: 0006368] | | |
| meiotic prophase I | 0.005644 | XRS2 DMC1 HOP2 RAD50 |
| [GO: 0007128] | | |
| nuclear | 0.006876 | NUP170 CHD1 SPT10 EST1 RSC2 NUP188 RAD50 HTZ1 EAF3 |
| organization and | | |
| biogenesis | | |
| [GO: 0006997] | | |
| meiosis I | 0.007223 | XRS2 DMC1 HOP2 RAD50 |
| [GO: 0007127] | | |
| DNA catabolism, | 0.007699 | XRS2 RAD50 |
| endonucleolytic | | |
| [GO: 0000737] | | |
| DNA catabolism | 0.007699 | XRS2 RAD50 |
| [GO: 0006308] | | |
| meiotic DNA | 0.007699 | XRS2 RAD50 |
| double-strand break | | |
| formation | | |
| [GO: 0042138] | | |

GO Cellular Component

| | | |
|---|---|---|
| meiotic | 0.000167 | XRS2 DMC1 HOP2 RAD50 |
| chromosome | | |
| [GO: 0005711] | | |
| transcription | 0.003945 | PAF1 CHD1 RTF1 |
| elongation factor | | |
| complex | | |
| [GO: 0008023] | | |
| chromosome | 0.00408 | RAD18 XRS2 DMC1 HOP2 EST1 RAD50 HTZ1 |
| [GO: 0005694] | | |

Deletions Consortium (Essentiality, Morphology)

| | | |
|---|---|---|
| Morphology - | 1.20E−09 | YBL006C SRO9 RAD18 XRS2 RPB9 PRP18 SRB2 RPN10 |
| Large | | RTT101 EST1 MMS22 RSC2 ZRC1 VPS75 RAD50 BFR1 CTF4 |
| Morphology - | 2.42E−09 | SRO9 CDC10 VPS64 XRS2 PRP18 SRB2 KEL1 SPT10 EST1 |
| Elongate | | MIH1 SPT21 YAF9 RAD50 VPS69 CLB2 CTF4 |
| Morphology - | 0.002755 | VPS64 LAG1 RPL27A ATP12 VPS75 WHI2 CLB2 |
| Football | | |
| Morphology - | 0.002844 | SRO9 SRB2 |
| branch | | |

Yeast Fitness Data

| | | |
|---|---|---|
| Slow Growers | 3.39E−05 | PAF1 XRS2 RPB9 PRP18 RPL27A SRB2 APQ12 SPT10 PFD1 |
| | | ATP12 YJR018W RPL14A EST1 MMS22 RSC2 SPT21 YAF9 |
| | | VPS75 RAD50 BFR1 RPL43A YPR044C VPS69 CTF4 VPS66 |

Large, 24 genes
MIPS Functional Classification

| | | |
|---|---|---|
| anion transporters | 0.002373 | CTP1 PHO87 |
| (Cl, SO4, PO4, | | |
| etc.) | | |

MIPS Phenotypes

| | | |
|---|---|---|
| Divalent cations | 0.000896 | CSG2 PHO87 |
| and heavy metals | | |
| resistance | | |
| Divalent cations | 0.002877 | CSG2 PHO87 SSD1 |
| and heavy metals | | |
| Trifluoperazine | 0.007009 | SSD1 |
| sensitivity | | |

GO Molecular Function

| | | |
|---|---|---|
| electrochemical | 0.000875 | AAC3 CTP1 PHO87 |
| potential-driven | | |
| transporter | | |
| [GO: 0015290] | | |
| porter | 0.000875 | AAC3 CTP1 PHO87 |
| [GO: 0015291] | | |
| tricarboxylate | 0.007009 | CTP1 |
| carrier | | |
| [GO: 0005371] | | |

GO Biological Process

| | | |
|---|---|---|
| anion transport | 0.003099 | CTP1 PHO87 |
| [GO: 0006820] | | |
| citrate transport | 0.00351 | CTP1 |
| [GO: 0015746] | | |
| mitochondrial | 0.00351 | CTP1 |
| citrate transport | | |
| [GO: 0006843] | | |
| tricarboxylic acid | 0.007009 | CTP1 |
| transport | | |
| [GO: 0006842] | | |

GO Cellular Component

| | | |
|---|---|---|
| prefoldin | 0.00351 | PFD1 |
| [GO: 0016272] | | |
| snRNA cap binding | 0.007009 | CBC2 |
| complex | | |
| [GO: 0005846] | | |

Deletions Consortium (Essentiality, Morphology)

| | | |
|---|---|---|
| Morphology - | 0.001454 | CDC10 YML117W YNL119W YOR073W |
| Elongate | | |
| Morphology - | 0.002042 | CTP1 RAD18 YNL119W YOR073W |
| Large | | |

Small, 95 genes
MIPS Functional Classification

| | | |
|---|---|---|
| mitochondrion | 0.007493 | MDM10 MSM1 OSM1 MGM101 MEF1 DIC1 NAM2 IMP1 |
| | | MTF1 MIP1 YPL183W-A YME1 |
| C-compound and | 0.007843 | PTC1 HXK1 LSC2 YHL012W PIG2 YUR1 SNF7 DIC1 GSF2 |
| carbohydrate | | HXT2 SIP3 OST3 YPL113C |
| metabolism | | |
| phosphate transport | 0.009073 | PHO90 DIC1 |

16/30 MIPS Subcellular Localization

| | | |
|---|---|---|
| mitochondria | 0.00449 | MDM10 MSM1 LSC2 OSM1 MGM101 MEF1 DIC1 NAM2 IMP1 |
| | | MTF1 MIP1 YPL183W-A YME1 |
| | | MIPS Protein Complexes |
| Proteases, | 0.005756 | IMP1 YME1 |
| mitochondrial | | |

Deletions Consortium (Essentiality, Morphology)

| | | |
|---|---|---|
| Morphology - WT | 1.01E−08 | MDM10 FUN26 EDS1 YCR090C OSH2 SED1 FIN1 RUB1 AHA1 |
| | | PEX10 YDR306C YDR326C SAC7 PHO4 HXK1 GCN1 |
| | | YGL242C YGR012W YGR212W LSC2 YHL012W YHL044W |
| | | SSP1 PIG2 YIL060W YIL087C YJL043W YJL083W PHO90 |
| | | OSM1 PTK2 MGM101 PRR1 YLL033W GTT2 SNF7 RPS0B |
| | | YLR049C XDJ1 YLR169W ECM38 DIC1 YLR405W YML030W |
| | | GSF2 YML131W YMR002W YMR003W HXT2 FMS1 RPS16A |
| | | IMP1 RPL36A MTF1 RCE1 SIP3 NGL1 ARG1 MOR1 CVT19 |
| | | YOR012W YOR021C RSB1 YOR072W ATX2 WHI5 YOR170W |
| | | YOR309C MIP1 GLR1 YPL113C MLH3 OYE3 YPL183W-A |
| | | PRM3 APL5 YPL267W SAM3 YME1 YPR091C |

Round, 14 genes
MIPS Functional Classification

| | | |
|---|---|---|
| allantoin and | 0.000165 | YLL055W YOR071C |
| allantoate | | |
| transporters | | |
| cell wall | 0.001495 | ECM33 HAL5 BUD8 |
| budding, cell | 0.005727 | MYO4 BUD8 DFG5 |
| polarity and | | |
| filament formation | | |
| fungal cell | 0.008339 | MYO4 ECM33 BUD8 DFG5 |
| differentiation | | |
| cell differentiation | 0.008339 | MYO4 ECM33 BUD8 DFG5 |

MIPS Phenotypes

| | | |
|---|---|---|
| Calcofluor white | 0.000871 | ECM33 HAL5 BUD8 |
| sensitivity | | |
| Hygromycin B | 0.001461 | ECM33 HAL5 |
| sensitivity | | |
| Calcofluor white | 0.001578 | ECM33 HAL5 BUD8 |
| Hygromycin B | 0.001695 | ECM33 HAL5 |
| Zymolyase | 0.002077 | ECM33 BUD8 |
| sensitivity | | |
| Pseudohyphae | 0.002077 | BUD8 DFG5 |
| formation | | |
| Cell wall mutants | 0.007245 | ECM33 HAL5 BUD8 |

MIPS Protein Classes

| | | |
|---|---|---|
| Class V | 0.004463 | MYO4 |

GO Molecular Function

| | | |
|---|---|---|
| endopolyphosphatase | 0.002234 | PPN1 |
| [GO: 0000298] | | |
| N-acetyl-gamma- | 0.004463 | SDL1 |
| glutamyl-phosphate | | |
| reductase | | |
| [GO: 0003942] | | |

GO Biological Process

| | | |
|---|---|---|
| pseudohyphal | 0.00473 | BUD8 DFG5 |
| growth | | |
| [GO: 0007124] | | |
| growth pattern | 0.00534 | BUD8 DFG5 |
| [GO: 0007150] | | |
| growth | 0.005766 | BUD8 DFG5 |
| [GO: 0040007] | | |
| polyphosphate | 0.006688 | PPN1 |
| metabolism | | |
| [GO: 0006797] | | |

Pointed, 98 genes
MIPS Phenotypes

| | | |
|---|---|---|
| Divalent cations | 4.03E−05 | PER1 SSD1 ZRT1 LHS1 YAP1 GTR1 QRI8 ROD1 |
| and heavy metals | | |
| Stress response | 0.000448 | GDH3 PER1 SSD1 ZRT1 LHS1 YAP1 GTR1 QRI8 ROD1 |
| defects | | |
| Divalent cations | 0.000628 | PER1 SSD1 ZRT1 GTR1 QRI8 ROD1 |
| and heavy metals | | |
| sensitivity | | |

MIPS Protein Complexes

| | | |
|---|---|---|
| Mitochondrial | 0.001472 | PET112 CBS2 SUV3 |
| translation | | |
| complexes | | |

GO Molecular Function

| | | |
|---|---|---|
| O-acetytransferase | 0.003485 | CAT2 MET2 |
| [GO: 0016413] | | |

GO Biological Process

| | | |
|---|---|---|
| response to drug | 0.000396 | KAP122 YALP1 PDR17 ROD1 |
| [GO: 0042493] | | |
| methionine | 0.001423 | CBF1 MET2 |
| biosynthesis | | |
| [GO: 0009086] | | |
| response to | 0.002257 | KAP122 YAP1 PDR17 ROD1 |
| chemical substance | | |
| [GO: 0042221] | | |
| methionine | 0.003485 | CBF1 SAM1 MET2 |
| metabolism | | |
| [GO: 0006555] | | |
| sulfur amino acid | 0.00483 | CBF1 MET2 |
| biosynthesis | | |
| [GO: 0000097] | | |
| response to | 0.006374 | PER1 LHS1 |
| unfolded protein | | |
| [GO: 0006986] | | |
| response to | 0.009765 | PER1 GRX4 KAP122 LHS1 STE11 YAP1 PDR17 ROD1 |
| external stimulus | | MF(ALPHA)1 |
| [GO: 0009605] | | |

Deletions Consortium (Essentiality, Morphology)

| | | |
|---|---|---|
| Morphology - WT | 3.42E−06 | GDH3 PIN4 PET112 SCO2 YBR281C APE3 MRPL32 YCR025C |
| | | YDR008C RAD28 CBS2 DON1 MUS81 HKR1 YDR428C |
| | | YEL015W UTR4 YER030W YGL231C ZRT1 RNH70 ECM29 |
| | | ARN2 FSH1 YHR112C YHR177W SDS3 MHP1 RPA34 |
| | | YJL207C YJL213W YJL215C CBF1 YJR087W YJR119C |
| | | YKL044W NUP100 LHS1 YKL137W YLR021W YLR123C |
| | | YLR128W YLR171W SAM1 UBC12 MID2 YAP1 MRPL39 |
| | | CAT2 HMG1 GTR1 QRI8 YMR107W YMR124W YMR153C-A |
| | | YMR157C YMR166C COX7 YMR262W YMR294W-A |
| | | YMR317W ADH6 SPS18 PDR17 YNR073C YOR012W ELG1 |
| | | YOR155C YOR223W PUS7 SPS4 GRD19 YPL159C |
| | | MF(ALPHA)1 YPL267W YPR022C YPR064W MRL1 MDM36 |

Clumped, 31 genes
GO Molecular Function

| | | |
|---|---|---|
| sterol C-24(28) | 0.004947 | ERG4 |
| reductase | | |
| [GO: 0000246] | | |
| kexin | 0.004947 | KEX2 |
| [GO: 0004290] | | |
| subtilase | 0.004947 | KEX2 |
| [GO: 0004289] | | |
| serine-type | 0.009869 | KEX2 |
| endopeptidase | | |
| [GO: 0004252] | | |
| pyruvate kinase | 0.009869 | PYK2 |
| [GO: 0004743] | | |

GO Biological Process

| | | |
|---|---|---|
| alpha-factor | 0.009869 | KEX2 |
| maturation | | |
| [GO: 0007326] | | |

Polarized bud growth, 5 genes
MIPS Functional Classification

| | | |
|---|---|---|
| budding, cell | 0.006932 | NAP1 BNI1 |
| polarity and | | |
| filament formation | | |

MIPS Phenotypes

| | | |
|---|---|---|
| Bud localization | 0.0003 | NAP1 BNI1 |
| Diamide resistance | 0.000798 | GRX1 |
| Menadione | 0.002392 | GRX1 |
| sensitivity | | |
| Diamide | 0.002392 | GRX1 |
| Menadione | 0.002392 | GRX1 |
| Budding mutants | 0.003501 | NAP1 BNI1 |

GO Molecular Function

| | | |
|---|---|---|
| caspase | 0.000798 | MCA1 |
| [GO: 0004199] | | |
| cysteine-type | 0.002392 | MCA1 |
| endopeptidase | | |
| [GO: 0004197] | | |
| cytoskeletal | 0.003984 | BNI1 |
| regulatory protein | | |
| binding | | |
| [GO: 0005519] | | |
| dolichyl- | 0.004779 | PMT5 |
| phosphate- | | |
| mannose-protein | | |
| mannosyltransferase | | |
| [GO: 0004169] | | |
| thiol-disulfide | 0.006368 | GRX1 |
| exchange | | |
| intermediate | | |
| [GO: 0030508] | | |
| protein binding | 0.009695 | NAP1 BNI1 |
| [GO: 0005515] | | |

GO Biological Process

| | | |
|---|---|---|
| cell death | 0.002392 | MCA1 |
| [GO: 0008219] | | |
| programmed cell | 0.002392 | MCA1 |
| death | | |
| [GO: 0012501] | | |
| apoptosis | 0.002392 | MCA1 |
| [GO: 0006915] | | |
| death | 0.002392 | MCA1 |
| [GO: 0016265] | | |
| nucleosome | 0.003188 | NAP1 |
| assembly | | |
| [GO: 0006334] | | |
| M phase of mitotic | 0.003444 | NAP1 BNI1 |
| cell cycle | | |
| [GO: 0000087] | | |
| mitotic spindle | 0.003984 | BNI1 |
| positioning and | | |
| orientation | | |
| [GO: 0040001] | | |
| mitotic spindle | 0.003984 | BNI1 |
| orientation | | |
| [GO: 0000132] | | |
| mRNA | 0.007162 | BNI1 |
| localization, | | |
| intracellular | | |
| [GO: 0008298] | | |
| redox homeostasis | 0.007955 | GRX1 |
| [GO: 0045454] | | |
| regulation of redox | 0.007955 | GRX1 |
| homeostasis | | |
| [GO: 0030503] | | |
| response to stress | 0.009418 | GRX1 BNI1 |
| [GO: 0006950] | | |

GO Cellular Component

| | | |
|---|---|---|
| polarisome | 0.003984 | BNI1 |
| [GO: 0000133] | | |
| | | Proteome Localization - Observed |
| cyto | 0.008252 | GRX1 PMT5 NAP1 MCA1 |

Other, 2 genes
MIPS Protein Complexes

| | | |
|---|---|---|
| Actin-associated | 0.007963 | SLA1 |
| proteins | | |

GO Biological Process

| | | |
|---|---|---|
| actin cortical patch | 0.003189 | SLA1 |
| assembly | | |
| [GO: 0000147] | | |

GO Cellular Component

| | | |
|---|---|---|
| actin cortical patch | 0.009552 | SLA1 |
| (sensu | | |
| Saccharomyces) | | |
| [GO: 0005857] | | |
| actin cortical patch | 0.009869 | SLA1 |
| (sensu Fungi) | | |
| [GO: 0030479] | | |

Budding, no genes w/ strong defects (total grader score >=8) found.
Pseudo-hyphal, no genes w/ strong defects (total grader score >=8) found.

TABLE 3
Alpha factor response mutants and control strains - ‘Slight’ shmoo mutants

| | | | | | Second | |
|---|---|---|---|---|---|---|
| | Common | Avg ratio of | From cell chip | First | retest | No ‘Shmoo’ |
| Gene | name | growth rates | (✓ = no shmoo) | retest | (stringent) | in >=2 of 3? |
| YNR068C | unannotated | 0.97 | ✓ | ✓ | ✓ | ✓ |
| YHR005C | GPA1 | 0.97 | ✓ | ✓ | ✓ | ✓ |
| YJR050W | ISY1 | 0.97 | ✓ | ✓ | low % shmoo | ✓ |
| YLR442C | SIR3 | 0.97 | ✓ | not included | ✓ | ✓ |
| YBR133C | HSL7 | 0.97 | ✓ | ✓ | low % shmoo | ✓ |
| YIL121W | QDR2 | 0.98 | ✓ | ✓ | low % shmoo | ✓ |
| YDL159W | STE7 | 0.98 | ✓ | ✓ | ✓ | ✓ |
| YOR147W | MDM32 | 0.98 | ✓ | ✓ | low % shmoo | ✓ |
| YDR462W | MRPL28 | 0.98 | ✓ | ✓ | ✓ | ✓ |
| YDL041W | unannotated | 0.99 | ✓ | ✓ | ✓ | ✓ |
| YDL042C | SIR2 | 0.99 | ✓ | ✓ | ✓ | ✓ |
| YER037W | PHM8 | 1.00 | ✓ | ✓ | low % shmoo | ✓ |
| YNL138W | SRV2 | 1.00 | few cell | ✓ | shmoo | NA |
| YDR461W | MFA1 | 1.00 | ✓ | ✓ | ✓ | ✓ |
| YEL004W | YEA4 | 1.00 | ✓ | ✓ | ✓ | ✓ |
| YEL072W | RMD6 | 1.00 | slight shmoo | | | NA |
| YOR212W | STE4 | 1.01 | ✓ | ✓ | ✓ | ✓ |
| YGL012W | ERG4 | 1.01 | ✓ | ✓ | ✓ | ✓ |
| YHL007C | STE20 | 1.01 | ✓ | ✓ | ✓ | ✓ |
| YDR103W | STE5 | 1.01 | ✓ | ✓ | ✓ | ✓ |
| YNL264C | PDR17 | 1.01 | ✓ | ✓ | shmoo | ✓ |
| YHR177W | GON3 | 1.01 | ✓ | ✓ | ✓ | ✓ |
| YPL006W | NCR1 | 1.01 | ✓ | ✓ | ✓ | ✓ |
| YIL011W | TIR3 | 1.02 | ✓ | ✓ | shmoo | ✓ |
| YDL073W | unannotated | 1.02 | ✓ | ✓ | ✓ | ✓ |
| YKR101W | SIR1 | 1.02 | ✓ | ✓ | shmoo | ✓ |
| YPL029W | SUV3 | 1.02 | ✓ | ✓ | ✓ | ✓ |
| YIL157C | FMP35 | 1.02 | ✓ | not included | not included | NA |
| YLR362W | STE11 | 1.02 | ✓ | shmoo | ✓ | ✓ |
| YIL069C | RPS24B | 1.03 | ✓ | ✓ | ✓ | ✓ |
| YPL049C | DIG1 | 1.03 | ✓ | ✓ | low % shmoo | ✓ |
| YOR369C | RPS12 | 1.04 | ✓ | ✓ | ✓ | ✓ |
| YIL049W | DFG10 | 1.04 | ✓ | ✓ | shmoo | ✓ |
| YIL084C | SDS3 | 1.05 | ✓ | ✓ | low % shmoo | ✓ |
| | | | | slight | | |
| YLR024C | UBR2 | 1.05 | slight shmoo | shmoo | shmoo | ✓ |
| YBR279W | PAF1 | 1.05 | ✓ | ✓ | low % shmoo | ✓ |
| YDR495C | VPS3 | 1.05 | ✓ | ✓ | ✓ | ✓ |
| YDR323C | PEP7 | 1.06 | ✓ | ✓ | slight shmoo | ✓ |
| YBR085W | AAC3 | 1.06 | ✓ | not included | ✓ | ✓ |
| YNL271C | BNI1 | 1.07 | ✓ | ✓ | shmoo | ✓ |
| YBL016W | Fus3 | 1.07 | ✓ | not included | shmoo | NA |
| YJL157C | FAR1 | 1.07 | ✓ | ✓ | low % shmoo | ✓ |
| YCL032W | STE50 | 1.08 | ✓ | ✓ | low % shmoo | ✓ |
| YIL047C | SYG1 | 1.08 | ✓ | ✓ | shmoo | ✓ |
| YDL181W | INH1 | 1.12 | ✓ | ✓(few cells) | ✓(few cells) | ✓ |
| YGL214W | unannotated | 1.13 | low % shmoo | shmoo | shmoo | |
| YNL016W | PUB1 | 1.18 | low % shmoo | shmoo | shmoo | |
| YOR036W | PEP12 | 1.19 | ✓ | ✓ | slight shmoo | ✓ |
| YAL023C | PMT2 | 1.19 | ✓ | shmoo | shmoo | |
| YJR152W | DAL5 | 1.19 | ✓ | ✓ | shmoo | ✓ |
| YPL161C | BEM4 | 1.20 | ✓ | ✓ | shmoo | ✓ |
| | | | | slight | | |
| YJL062W | LAS21 | 1.25 | ✓ | shmoo | shmoo | ✓ |
| YOR085W | OST3 | 1.25 | slight shmoo | shmoo | shmoo | |
| YER149C | PEA2 | 1.29 | low % shmoo | shmoo | shmoo | |
| YCL027W | FUS1 | 1.29 | slight shmoo | shmoo | shmoo | |
| YAL027W | Unannotated | 1.36 | low % shmoo | shmoo | shmoo | |
| YMR123W | PKR1 | 1.37 | shmoo(few cell) | shmoo | shmoo | |
| YAL034C | FUN19 | 1.38 | ✓ | shmoo | shmoo | |
| YAL031C | FUN21 | 1.41 | low % shmoo | shmoo | shmoo | |
| YLR418C | CDC73 | 1.42 | slight shmoo | shmoo | shmoo | |
| YPR023C | EAF3 | 1.42 | ✓ | ✓ | shmoo | ✓ |
| YMR274C | RCE1 | 1.43 | low % shmoo | not included | not included | NA |
| YAL044C | GCV3 | 1.48 | slight shmoo | shmoo | shmoo | |
| YPL120W | VPS30 | 1.49 | slight shmoo | shmoo | shmoo | |
| YER119C | AVT6 | 1.49 | slight shmoo | shmoo | shmoo | |
| YAL053W | Unannotated | 1.50 | slight shmoo | shmoo | shmoo | |
| YNL094W | APP1 | 1.51 | ✓ | shmoo | shmoo | |
| YPL259C | APM1 | 1.51 | ✓(few cell) | shmoo | shmoo | |
| YBR276C | PPS1 | 1.52 | ✓ | shmoo | shmoo | |
| YIL052C | RPL34B | 1.52 | low % shmoo | not included | not included | NA |
| YGL211W | Unannotated | 1.52 | slight shmoo | shmoo | shmoo | |
| YML131W | Unannotated | 1.52 | slight shmoo | shmoo | shmoo | |
| YOR069W | VPS5 | 1.53 | slight shmoo | shmoo | shmoo | |
| YOR015W | Unannotated | 1.55 | few cells | shmoo | shmoo | |
| YKL007W | CAP1 | 1.56 | low % shmoo | shmoo | shmoo | |
| YLL021W | SPA2 | 1.56 | low % shmoo | shmoo | shmoo | |
| YER056C | FCY2 | 1.57 | ✓ | shmoo | shmoo | |
| YBR250W | Unannotated | 1.61 | low % shmoo | shmoo | shmoo | |
| YNL023C | FAP1 | 1.61 | slight shmoo | shmoo | shmoo | |
| YJL051W | unannotated | 1.62 | low % shmoo | shmoo | shmoo | |
| | | | | slight | | |
| YAL002W | VPS8 | 1.63 | ✓ | shmoo | low % shmoo | ✓ |
| | | | | slight | | |
| YCL008C | STP22 | 1.65 | slight shmoo | shmoo | shmoo | ✓ |
| YCR030C | SYP1 | 1.65 | slight shmoo | shmoo | shmoo | |
| YPL250C | ICY2 | 1.67 | slight shmoo | shmoo | shmoo | |
| YOR076C | SKI7 | 1.67 | few cells | shmoo | shmoo | |
| YOR044W | Unannotated | 1.68 | few cells | shmoo | shmoo | |
| YOR019W | Unannotated | 1.68 | few cells | shmoo | shmoo | |
| | | | | low % | | |
| YOR089C | VPS21 | 1.69 | ✓ | shmoo | shmoo | ✓ |
| YLR013W | GAT3 | 1.70 | slight shmoo | no image | shmoo | NA |
| YNR019W | ARE2 | 1.70 | slight shmoo | shmoo | shmoo | |
| YOL079W | Unannotated | 1.70 | slight shmoo | shmoo | shmoo | |
| YLR079W | SIC1 | 1.70 | slight shmoo | shmoo | shmoo | |
| YKL212W | To redo | 1.70 | ✓ | shmoo | shmoo | |
| YPR060C | ARO7 | 1.72 | ✓ | shmoo | shmoo | |
| YPL155C | KIP2 | 1.73 | low % shmoo | shmoo | shmoo | |
| | | | | slight | | |
| YPL002C | SNF8 | 1.77 | slight shmoo | shmoo | shmoo | ✓ |
| YLL024C | SSA2 | 1.79 | slight shmoo | shmoo | shmoo | |
| YCR062W | unannotated | 1.79 | slight shmoo | shmoo | shmoo | |
| YBL098W | BNA4 | 1.80 | slight shmoo | shmoo | shmoo | |
| YLR065C | Unannotated | 1.81 | slight shmoo | shmoo | shmoo | |
| YML095C-A | Unannotated | 1.81 | slight shmoo | shmoo | shmoo | |
| YLR338W | YLR338W | 1.81 | ✓ | shmoo | shmoo | |
| YDR357C | Unannotated | 1.81 | slight shmoo | shmoo | shmoo | |
| | | | | slight | | |
| YPL065W | VPS28 | 1.82 | slight shmoo | shmoo | shmoo | ✓ |
| YMR160W | unannotated | 1.82 | slight shmoo | shmoo | shmoo | |
| YBR134W | unannotated | 1.82 | ✓ | shmoo | shmoo | |
| YBL101C | ECM21 | 1.83 | slight shmoo | shmoo | shmoo | |
| YDR525W | API2 | 1.84 | ✓ | shmoo | shmoo | |
| YJR109C | CPA2 | 1.84 | slight shmoo | shmoo | shmoo | |
| YAL026C | DRS2 | 1.86 | slight shmoo | shmoo | shmoo | |
| YBR078W | ECM33 | 1.86 | ✓ | ✓ | shmoo | ✓ |
| YML094W | GIM5 | 1.87 | shmoo | shmoo | shmoo | |
| YOR342C | Unannotated | 1.87 | ✓ | shmoo | shmoo | |
| | | | | low % | | |
| YLR417W | VPS36 | 1.89 | slight shmoo | shmoo | shmoo | ✓ |
| YGL020C | MDM39 | 1.90 | low % shmoo | shmoo | shmoo | |
| YGL151W | NUT1 | 1.91 | few cell | shmoo | shmoo | |
| YAR044W | OSH1/SWH1 | 1.93 | slight shmoo | shmoo | shmoo | |
| YJR075W | HOC1 | 1.93 | low % shmoo | shmoo | shmoo | |
| YNL079C | TPM1 | 1.99 | slight shmoo | not included | shmoo | NA |
| YBR072W | HSP26 | 2.05 | slight shmoo | shmoo | shmoo | |
| YDR511W | ACN9 | 2.11 | slight shmoo | shmoo | shmoo | |
| YBL027W | RPL19B | 2.15 | low % shmoo | shmoo | shmoo | |
| YBR074W | unannotated | 2.20 | slight shmoo | shmoo | shmoo | |
| YOL036W | Unannotated | 2.21 | slight shmoo | shmoo | shmoo | |
| YDL006W | PTC1 | 2.22 | slight shmoo | shmoo | shmoo | |
| YLR110C | CCW12 | 2.23 | slight shmoo | shmoo | shmoo | |
| YOR039W | CKB2 | 2.26 | low % shmoo | shmoo | shmoo | |
| YDR132C | Unannotated | 2.26 | slight shmoo | shmoo | shmoo | |
| YBR191W | RPL21A | 2.27 | ✓ | shmoo | shmoo | |
| YDL176W | unannotated | 2.30 | slight shmoo | shmoo | shmoo | |
| YAR003W | SWD1 | 2.30 | slight shmoo | shmoo | shmoo | |
| YLR025W | SNF7 | 2.32 | ✓ | no image | shmoo | NA |
| YDR484W | VPS52 | 2.41 | slight shmoo | shmoo | shmoo | |
| YNL129W | unannotated | 2.50 | slight shmoo | shmoo | shmoo | |
| | | | | no good | | |
| YJR032W | CPR7 | 2.57 | ✓ | image | shmoo | NA |
| YER111C | SWI4 | 2.61 | slight shmoo | shmoo | shmoo | |
| | | | | low % | | |
| YGL148W | ARO2 | 2.78 | ✓ | shmoo | NA | NA |
| YDR127W | ARO1 | 2.79 | ✓ | shmoo | shmoo | |
| YDR128W | Unannotated | 2.94 | slight shmoo | not included | shmoo | NA |
| YNL083W | unannotated | 3.05 | slight shmoo | shmoo | shmoo | |
| YAL013W | DEP1 | 5.34 | ✓ | shmoo | shmoo | |
| YCR081W | SRB8 | 25.44 | low % shmoo | shmoo | shmoo | |

142 KO strains identified via cell chip, with measured extent of growth arrest & shmoo defect reproducibility

In operation, the present invention has also been used by fixing and spheroplasting yeast for cell chip-based immunoassays and fluorescent in situ hybridization (FISH) assays. This work addresses the use of spotted cell microarrays for measuring protein and RNA expression levels and sub-cellular localization. The cell chips were constructed from fixed and spheroplasted yeast cells and probed with, e.g., fluorescently tagged probes against specific proteins. Next, highly parallelized immunoassay were performed across the entire cell chip, in order to measure the expression level and localization of the probe target as a function of a comprehensive set of genetic backgrounds. For example, probing cell chips constructed from the yeast deletion library with a fluorescent probe specific to actin would show both the localization and expression-level response of the actin cytoskeleton's structure to the loss of each of the ˜4,800 deleted genes.

The cell chips of the present invention were particularly useful by separating the cell growth stage from the imaging portion of the assay, e.g., immunomicroscopy in 96 well plates that permits short and long term analysis of cells and the data. Thus, the present invention allows the user to create essentially identical cell chips from the same set of cells, e.g., printing up to 200 slides in a single session from a set of yeast strains grown in 96-well plates. As each slide can be probed with a different fluorescent probe, this greatly simplifies high-throughput immunoassays. For example, probing the yeast deletion library in 96-well plates (roughly 50 plates/copy of the library) with 50 different antibodies would take a tremendous amount of labor to manipulate the required minimum of 2,500 96-well plates. However, this same set of experiments, even given its grand scale, is entirely feasible with cell chips, requiring at a minimum only 50 microscope slides generated from a single copy of the library, easily generated in a single day. The imaging of these slides, while slow (˜1 month), is far more automated than that of 96 well plates. Also, the cell chip version would be very well controlled for variations in cell growth—all of the slides would be generated from the same set of cell cultures.

The cell chips or microarrays of the present invention were also used to visualize the localization of microtubules in cells by probing cell chips with an antibody against tubulin. The ability of the cell printing of spheroplasted yeast cells was verified and the cell chips were probed with antibodies. S. cerevisiae cells were first fixed with formaldehyde, spheroplasted, and then printed as microarrays on to poly-L-lysine coated slides. Cells on the chip were permeabilized in cold methanol for 5 min then in cold acetone. Cell chips were probed with Rat anti-alpha tubulin (YOL1/34; Serotec) as the primary antibody, followed by an FITC conjugated goat anti-rat 1 gG secondary antibody (Serotec). After washing, the cell chip was additionally probed with DAPI and imaged as before. FIG. 11 shows the distinct microtubule morphology obtained using the anti-tubulin antibody, demonstrating that it is possible to perform high-throughput detection of proteins in cells on a cell chip by probing with a specific probe.

The present invention has also been used in two separate spheroplast cell arrays. First, yeast were fixed and spheroplasted in 96 well plates, then printed as cell microarrays and used for immunoassays. The slides were stable for at least 1 month. Using a previously printed slide of fixed cells the present invention allowed for the detection of spheroplast cells directly on the microarray. This approach is both technically simpler and more consistent, as it imposes uniform spheroplasting conditions for all strains.

FIG. 13 shows images from both approaches. The left panel shows spheroplasted S. cerevisiae cells printed onto poly-L-lysine coated glass slides and probed with an antibody for microtubules (MT), which are visible as filaments in the control strain but defective in the bim1Δ strain (conditional on 2 hours 37° treatment). For this cell chip or microarray, ˜300 yeast deletion strains (BY4741 background) were used, and each strain had two replicates. Spots are ˜200 μm in diameter, separated by 410 μm. The right panel shows results from spheroplasting directly on a pre-printed yeast deletion cell array (constructed from the entire yeast deletion strain collection) and probing with concanavalin-A-TRITC to image cell wall integrity. This method appears to offer relatively fine control, as shown by the partial perforation of treated cells' walls after mild spheroplasting.

The cell chips were also used for fluorescent in situ hybridization (FISH) assays to determine RNA expression levels and localization using spotted cell microarrays. FIGS. 13A to 13D show yeast cells fixed with formaldehyde, spheroplasted, and printed on a cell microarray. Printed cells were permeabilized and probed for expression of the U3 small nucleolar RNA (snoRNA) by use of a Cy5-labeled nucleotide complementary to the U3 snoRNA, following a published FISH protocol (Hieronymus, H., Yu, M. C., & Silver, P. A. (2004) Genome-wide MRNA surveillance is coupled to mRNA export, Genes Dev 18: 2652-2662, relevant portions incorporated herein by reference). After washing, the cell chip was additionally probed with DAPI and imaged. FIG. 13A is a differential interference contrast (DIC) image of a field of cells on the microarray. FIG. 13B is a DAPI fluorescence image of DNA locations for the same cells shown in FIG. 13A. FIG. 13C is an image of Cy5 fluorescence, corresponding to location of U3 snoRNA in the cells. FIG. 13D shows the overlay of the images in FIG. 13A to FIG. 13C, confirming the nucleolar location of the Cy5 signal (Beltrame, M. & Tollervey, D. (1995) Base pairing between U3 and the pre-ribosomal RNA is required for 18S rRNA synthesis, EMBO J. 14:4350-6).

Cell chips therefore provide a method for detecting and measuring RNA as well as protein within the same cell. The feasibility of FISH on the complete yeast deletion library has been recently shown (Hieronymus, supra).

The present invention also includes an apparatus and method for assembling and imaging spotted cell microarrays of the full collection of ˜4,100 green fluorescent protein (GFP)-tagged yeast strains. A GFP)-tagged yeast strain for use with the present invention may include on such as that taught by Huh, W. K. et al. (2003) Global analysis of protein localization in budding yeast, Nature 425: 686-691, relevant portions incorporated herein by reference. Systematically mapping protein localization becomes practical through the construction of cell microarrays from the GFP-tagged yeast strain collection. This is an arrayed library of yeast strains expressing full-length, chromosomally tagged green fluorescent protein (Chalfie, M., Tu, Y., Euskirchen, G., Ward, W. W., & Prasher, D. C. (1994) Green fluorescent protein as a marker for gene expression, Science 263: 802-805, relevant portions incorporated herein by reference) fusion proteins that enables nearly every protein (˜4,100 proteins) to be monitored. A GFP-tagged strain collection was used for these studies (Invitrogen, USA) (see http://yeastgfp.ucsf.edu/).

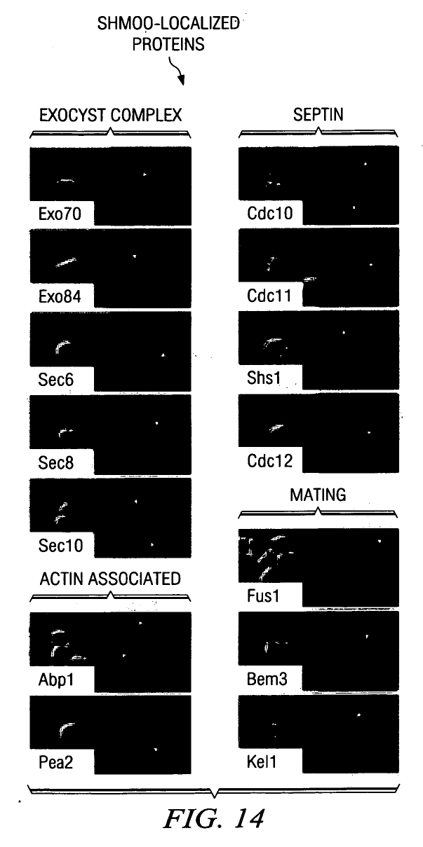

The cell chips were also imaged for a full GFP library treated with alpha factor. The GFP-fusion library was treated with the mating pheromone alpha factor and the cells fixed with formaldehyde. The cell chips were printed and then imaged. By visual inspection, the change in sub-cellular localization of each of the ˜4,100 GFP-tagged proteins was analyzed in parallel, providing a comprehensive measurement of protein localization changes accompanying morphological reorganization. A major application of the cell chips is that of measuring condition-specific spatial remodeling of proteins. In initial results, >120 proteins appear to change location or expression levels in a pheromone-dependent manner, judged by examination of the image sets by 2 graders. The proteins are strongly enriched for cytoskeletal proteins (actin, septin), proteins in polarized growth and exocytosis, and for systems physically linked to the shmoo tip (e.g., the spindle pole body). Many known shmoo-tip localized proteins are correctly found (e.g., Fus1, Rvs167, Smy1, Cbk1). Although the data are preliminary and require separate validation, it appears that one new system we localize to the growing shmoo tip is the exocyst, a protein complex known to be required for exocytosis and polarized growth during budding and mother/daughter cell division (TerBush, D. R. et al. (1996) The Exocyst is a multiprotein complex required for exocytosis in Sacciharomyces cerevisiae, EMBO J. 15: 6483-94), but not known to be associated with the mating projection. Example shmoo tip and base-localized proteins found in this screen are shown in FIG. 14, showing DIC and GFP-fluorescence images of pheromone-treated cells from the indicated GFP-tagged strains.

Use of spotted cell microarrays with other organisms. The present invention was also used with prokaryotic cells. E. coli bacteria were printed using the same approach as for yeast cells (supra). A sample microarray is shown below in part A, with magnification of two of the spots in part B. These particular cells of E. coli express a recombinant fusion protein between the Lactococcus lactis L1.LtrB group II intron-encoded reverse transcriptase (LtrA) protein and green fluorescent protein (GFP). This particular protein is localized to the poles of the cells (Zhao, J. & Lambowitz A. M. (2005) Inaugural Article: A bacterial group II intron-encoded reverse transcriptase localizes to cellular poles, Proc Natl Acad Sci USA 102:16133-40), as can be seen in the GFP epifluorescence images on the right hand side of panel B, corresponding to the same cells picture in the left hand side of panel B.

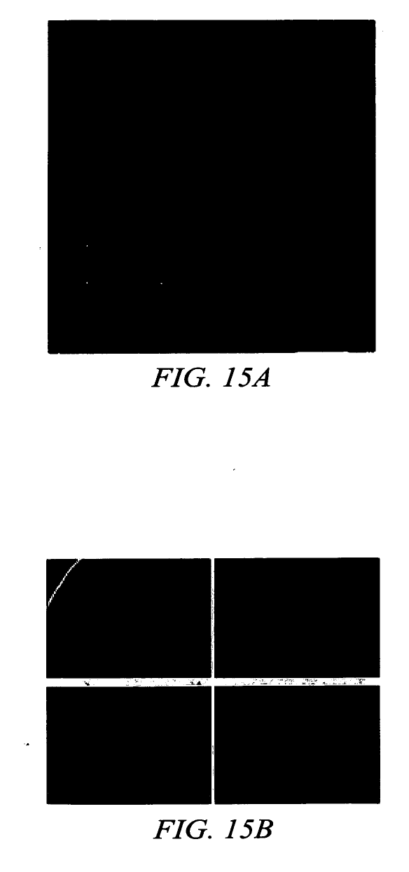

FIGS. 15A and 15B are examples of Escherichia coli spotted cell microarrays (cell chips). E. coli mutant strains were generated via mariner transposon insertional mutagenesis (strains were constructed by and are courtesy of Junhua Zhao and Alan Lambowitz, University of Texas at Austin) and spotted onto poly L-lysine coated glass slides. These strains are from a collection of ˜5,000 deletion strains in the HMS174 (DE3) genetic background (F⁻ recA1 hsdR (τ_{K12}⁻m_{K12}⁺) Rif^{R} (DE3)). FIG. 15A is an image of a small-scale E. coli spotted cell microarray. For this proof-of-concept experiment, the E. coli cell microarrays were constructed from 48 E. coli deletion strains, each strain spotted multiple times. Each strain carried a plasmid encoding an LtrA-GFP fusion protein. Spots are ˜200 μm in diameter, separated by 600 μm. FIG. 15B shows s a sample image that shows close-up view of arrayed E. coli cells. Left panel shows the DIC images and right panel shows the corresponding images of GFP-fused proteins which are normally localized at the two poles of each cell.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification, but only by the claims.

### REFERENCES

Winzeler, E. A., et al., Functional characterization of the S. cerevisiae genome by gene deletion and parallel analysis. Science 285, 901-6 (1999).
Fields, S., Pheromone response in yeast. Trends Biochem Sci 15, 270-3 (1990).
Elion, E. A., Pheromone response, mating and cell biology. Curr Opin Microbiol 3, 573-81 (2000).
Gad, M., A., Itoh, and A. Ikai Mapping cell wall polysaccharides of living microbial cells using atomic force microscopy. Cell Biol Int 21: 697-706 (1997).
Stuart, K. R. and E. S. Cole Nuclear and cytoskeletal fluorescence microscopy techniques. Methods Cell Biol 62: 291-311 (2000).
Poot, M., et al., Analysis of mitochondrial morphology and function with novel fixable fluorescent stains. J Histochem Cytochem 44: 1363-1372 (1996).
Vida, T. A. and S. D. Emr A new vital stain for visualizing vacuolar membrane dynamics and endocytosis in yeast. J Cell Biol 128: 779-792 (1995).
Bonangelino, C. J., E. M. Chavez, and J. S. Bonifacino Genomic screen for vacuolar protein sorting genes in Saccharomyces cerevisiae. Mol Biol Cell 13: 2486-2501(2002).
Giaever, G. et al., Functional profiling of the Saccharomyces cerevisiae genome. Nature 418, 387-91 (2002).
Nishizuka, S., et al., Proteomic profiling of the NCI-60 cancer cell lines using new high-density reverse-phase lysate microarrays. Proc Natl Acad Sci U S A 100, 14229-34 (2003).
Warringer, J., Ericson, E., Fernandez, L., Nerman, O. & Blomberg, A., High-resolution yeast phenomics resolves different physiological featuresin the saline response. Proc Natl Acad Sci U S A 100, 15724-9 (2003).
Bochner, B. R., Gadzinski, P. & Panomitros, E., Phenotype microarrays for high-throughput phenotypic testing and assay of gene function. Genome Res 11, 1246-55 (2001).
Saito, T. L. et al., SCMD: Saccharomyces cerevisiae Morphological Database. Nucleic Acids Res 32 Database issue, D319-22 (2004).
Xu, C. W. High-density cell microarrays for parallel functional determinations. Genome Res 12, 482-6 (2002).
Wulfkuhle, J. D. et al. Signal pathway profiling of ovarian cancer from human tissue specimens using reverse-phase protein microarrays. Proteomics 3, 2085-90 (2003).
Schwenk, J. M., Stoll, D., Templin, M. F. & Joos, T. O., Cell microarrays: an emerging technology for the characterization of antibodies. Biotechniques Suppl, 54-61 (2002).
Kononen, J. et al., Tissue microarrays for high-throughput molecular profiling of tumor specimens. Nat Med 4, 844-7 (1998).
Ziauddin, J. & Sabatini, D. M. Microarrays of cells expressing defined cDNAs. Nature 411, 107-10 (2001).
Silva, J. M., Mizuno, H., Brady, A., Lucito, R. & Hannon, G. J., RNA interference microarrays: high-throughput loss-of-function genetics in mammalian cells. Proc Natl Acad Sci USA 101, 6548-52 (2004).
DeRisi, J. L., Iyer, V. R. & Brown, P. O., Exploring the metabolic and genetic control of gene expression on a genomic scale. Science 278, 680-6 (1997).
Dohlman, H. G. & Thomer, J. W., Regulation of G protein-initiated signal transduction in yeast: paradigms and principles. Annu Rev Biochem 70, 703-54 (2001).
Roberts, C. J., et al., Signaling and circuitry of multiple MAPK pathways revealed by a matrix of global gene expression profiles. Science 287, 873-80 (2000).
Katzmann, D. J., Odorizzi, G. & Emr, S. D., Receptor downregulation and multivesicular-body sorting. Nat Rev Mol Cell Biol 3, 893-905 (2002).
Dulic, V. & Riezman, H., Saccharomyces cerevisiae mutants lacking a functional vacuole are defective for aspects of the pheromone response. J Cell Sci 97 ( Pt 3), 517-25 (1990).
Chen, L. & Davis, N. G., Recycling of the yeast a-factor receptor. J Cell Biol 151, 731-8 (2000).
Malathi, K. et al. Mutagenesis of the putative sterol-sensing domain of yeast Niemann Pick C-related protein reveals a primordial role in subcellular sphingolipid distribution. J Cell Biol 164, 547-56 (2004).
van den Hazel, H. B., et al. PDR16 and PDR17, two homologous genes of Saccharomyces cerevisiae, affect lipid biosynthesis and resistance to multiple drugs. J Biol Chem 274, 1934-41 (1999).
Benachour, A., et al., Deletion of GP17, a yeast gene required for addition of a side chain to the glycosylphosphatidylinositol (GPI) core structure, affects GPI protein transport, remodeling, and cell wall integrity. J Biol Chem 274, 15251-61 (1999).
Zweytick, D., Hrastnik, C., Kohlwein, S. D. & Daum, G., Biochemical characterization and subcellular localization of the sterol C-24(28) reductase, erg4p, from the yeast saccharomyces cerevisiae. FEBS Lett 470, 83-7 (2000).
Bagnat, M. & Simons, K., Cell surface polarization during yeast mating. Proc Natl Acad Sci USA 99, 14183-8 (2002).
Lee, I., Date, S. V., Adai, A. T. & Marcotte, E. M. A probabilistic functional network of yeast genes. (submitted) (2004).
Chang, M., et al., A complex containing RNA polymerase II, Paf1p, Cdc73p, Hpr1p, and Ccr4p plays a role in protein kinase C signaling. Mol Cell Biol 19, 1056-67 (1999).
Mueller, C. L., Porter, S. E., Hoffinan, M. G. & Jaehning, J. A. The Pafl complex has functions independent of actively transcribing RNA polymerase II. Mol Cell 14, 447-56 (2004).
Dahan, O. & Kupiec, M., Mutations in genes of Saccharomyces cerevisiae encoding pre-mRNA splicing factors cause cell cycle arrest through activation of the spindle checkpoint. Nucleic Acids Res 30, 4361-70(2002).
Ni, L. & Snyder, M., A genomic study of the bipolar bud site selection pattern in Saccharomyces cerevisiae. Mol Biol Cell 12, 2147-70 (2001).
Huh, W. K., et al., Global analysis of protein localization in budding yeast. Nature 425, 686-91 (2003).
Fraser, A. G. & Marcotte, E. M., A probabilistic view of gene function. Nat Genet 36, 559-64 (2004).
Mosch, H. U. & Fink, G. R., Dissection of filamentous growth by transposon mutagenesis in Saccharomyces cerevisiae. Genetics 145, 671-84 (1997).
Beltrame, M. & Tollervey, D. (1995) Base pairing between U3 and the pre-ribosomal RNA is required for 18S rRNA synthesis, EMBO J. 14:4350-6.

What is claimed is:

1. A method of analyzing one or more cell characteristics comprising the steps of:
depositing one or more spots on a substrate, wherein the two or more spots comprise a suspension of one or more cells from a strain collection; and
evaluating optically the one or more cells after the cells have been contacted with one or more agents.

2. The method of claim 1, wherein the strain collection comprises a haploid deletion strain collection.

3. The method of claim 1, wherein the strain collection comprising a haploid deletion strain collection in which each strain in the collection lacks the coding sequence of one or more genes.

4. The method of claim 1, wherein the two or more spots comprise between about 5-10, 10-20, 20-40, 40-50, 50-60, 70-80 or 80-100 cells.

5. The method of claim 1, wherein the one or more spots are between about 100 and 300 μm in diameter.

6. The method of claim 1, further comprising the step of taking a control imaging.

7. The method of claim 1, further comprising the step of taking a control imaging, wherein the control image is an image of the one or more cells before contacting the one or more cells with the one or more agents.

8. The method of claim 1, wherein the step of evaluating optically includes observing one or more of the following: light intensity, fluorescence lifetime, polarization, wavelength shift, wavelength emission wavelength adsorption, chemiluminescence emission, FRET emission, ELISA emissions or a combination thereof.

9. The method of claim 1, wherein the step of evaluating optically includes observing one or more of the following cellular characteristics: the cellular morphology, the cellular physiology, the cell shape, the cell structure, the presence and/or location of a component, the presence and/or location of a nucleic acid sequence or combinations thereof.

10. The method of claim 1, wherein the step of evaluating optically includes recording one or more properties of the one or more cells.

11. The method of claim 10, wherein the one or more properties is a differential interference contrast spectrum, a fluorescence wavelength, a visible wavelength, a cellular morphology, cellular biochemistry or a combination thereof.

12. The method of claim 1, wherein the step of imaging includes recording one or more images using a digital signal, an analogue signal or a combination thereof.

13. The method of claim 1, wherein the one or more agents affects one or more pathways selected from metabolic, epigenetic, genetic, signal transduction, transcription, transfection, replication, mitosis, meiosis, intracellular transport, extracellular transport, cytoskeletal, oxidative phosphorylation, phosphorylation, locomotion, phagocytosis, RNAi or a combination thereof.

14. The method of claim 1, wherein the one or more agents includes vitamins, minerals, small molecules, drugs, test compounds, antibodies, probes, stains, nucleic acid stains, antibody stains, affinity reagents or combinations thereof.

15. The method of claim 1, wherein the step of evaluating optically one or more cells includes the step of analyzing one or more cellular features in one or more genetic backgrounds.

16. The method of claim 1, wherein the step of evaluating optically includes observing one or more of the following: light intensity, fluorescence lifetime, polarization, wavelength shift or combinations thereof.

17. The method of claim 1, wherein the step of evaluating optically includes observing one or more of the following cell characteristics: cell morphology, cell physiology, the cell shape, cell structure, the presence of a component or a combination thereof.

18. The method of claim 1, wherein the cells are selected from prokaryotic, archaebacteria, and eukaryotic cells.

19. A method of analyzing the localization of one or more proteins comprising the steps of:
depositing a suspension of one or more cells on a substrate, wherein the one or more cells each comprise a clonal population from a strain collection;
imaging the one or more cells; and
analyzing one or more characteristic of the one or more proteins of the one or more cells.

20. An automated cell analyzing system comprising:
a substrate, wherein the substrate comprises one or more locations on which one or more spots may be deposited thereon, wherein the one or more spots include a suspension of one or more cells from a strain collection;
an optical system; and
a recording system.

21. A method for identifying a pattern of one or more cellular responses attributable to an a substance comprising:
depositing one or more spots on a substrate, wherein the one or more spots include a suspension of one or more cells from a strain collection;
imaging the one or more spots;
identifying one or more cellular responses exhibited by the one or more cells;
contacting the one or more cells with a substance;
imaging the one or more spots;
identifying the one or more cellular responses exhibited by the one or more cells contacted with the substance; and
comparing the one or more cellular responses exhibited by the one or more cells before the step of contacting and the one or more cellular responses exhibited by the one or more cells after the step of contacting, wherein the pattern of one or more cellular responses attributable to the substance is identified.

22. A method of content-based image retrieval comprising the steps of:
extracting a one or more descriptive features from each of one or more images that represent cell characteristics, the one or more images obtained with one or more different imaging tools;
recording the one or more descriptive features to form a image collection;
indexing the image collection to produce a searchable database, the imaging method including one or more set of groups based on similar image content;
extracting a query image to be characterized, the query image comprising the one or more descriptive features; and
retrieving one or more candidate images from the searchable database based on an image similarity criterion to the one or more descriptive features of the query image.

23. A cellular microarray for examining cellular characteristics comprising:
an array of two or more cells deposited on a substrate, wherein the two or more cells are from a strain collection; and
one or more agents placed into contact with the two or more cells, wherein one or more cellular characteristics produced as a result of the one or more agents placed in contact with the one or more cells is evaluated.

24. The cellular microarray claim 23, wherein the strain collection comprises a haploid deletion strain collection.

25. The cellular microarray of claim 23, wherein the substrate is a glass slides coated with poly L-lysine, ConA, Mn²⁺, Ca²⁺, biotin, streptavidin, antibodies, carbohydrates, lectins or a combination thereof.

26. The cellular microarray of claim 23, wherein the strain collection is a collection of strains, wherein each strain in the collection lacks the coding sequence of one or more genes.

27. A yeast cellular microarray collection comprising one or more spots deposited on a substrate, wherein each of the one or more spots include a unique suspension of one or more haploid deletion yeast cells from a strain collection, wherein the location of each one or more haploid deletion cells are known.

28. A method for identifying a pattern of one or more cellular responses attributable to an a substance comprising:
depositing one or more spots on a substrate, wherein the one or more spots include a suspension of one or more human cells from a strain collection;
imaging the one or more spots;
identifying one or more cellular responses exhibited by the one or more human cells;
contacting the one or more human cells with a substance;
imaging the one or more spots;
identifying the one or more cellular responses exhibited by the one or more human cells contacted with the substance; and
comparing the one or more cellular responses exhibited by the one or more human cells before the step of contacting and the one or more cellular responses exhibited by the one or more human cells after the step of contacting, wherein the pattern of one or more cellular responses attributable to the substance is identified.
